(19) United States
(12) Patent Application Publication
(10) Pub. No.: US 2026/0266834 A1
(43) Pub. Date: Sep. 10, 2026

(54) Compositions And Methods For Detecting Proteins In Protein Corona

(71) Applicant: Board of Trustees of Michigan State University, East Lansing, MI (US)

(72) Inventors: Morteza MAHMOUDI, East Lansing, MI (US); Amirata Saei DIBAVAR, Solna (SE)

(21) Appl. No.: 19/489,183

(22) PCT Filed: May 31, 2024
(86) PCT No.: PCT/US2024/031979

§ 371 Date: Dec. 1, 2025

Related U.S. Application Data
(60) Provisional application No. 63/471,578, filed on Jun. 7, 2023.
(60) Provisional application No. 63/603,325, filed on Nov. 28, 2023.

(51) Int. Cl. G01N 33/68

(52) U.S. Cl. CPC G01N 33/6848; G01N 33/6887; G01N 33/6896; G01N 2333/76; G01N 2333/775; G01N 2333/79; G01N 2405/04; G01N 2800/28; G01N 2800/32

(57) ABSTRACT

A method for detecting proteins and/or their proteoforms is provided herein that includes adding one or more small molecule and one or more protein-binding agent to a biological sample, where a protein corona is formed on the surface of the protein-binding agent, and the proteins and/or their proteoforms in the protein corona are detected by an antibody-based technique or a proteomics technique. Also provided herein is a method for detecting one or more biomarker or a pattern of one or more biomarker associated with a disease or health spectrum condition, as well as a method of diagnosing or predicting a disease in a subject. Compositions including one or more small molecule, one or more protein-binding agent, and one or more biological sample are also provided.

### CROSS-REFERENCE TO RELATED PATENT APPLICATIONS

This application claims the benefit of U.S. Provisional Patent Application No. 63/471,578 filed on 7 Jun. 2023 and U.S. Provisional Application No. 63/603,325 filed on 28 Nov. 2023. The entire content of each patent application recited above is hereby incorporated by reference.

### GOVERNMENT SUPPORT STATEMENT

This invention was made with government support under DK131417 awarded by the U.S. National Institute of Diabetes and Digestive and Kidney Diseases. The government has certain rights in the invention.

### FIELD

This disclosure generally relates to a method of adding small molecules to biological samples for enhancement of proteome analysis of protein corona at the surface of protein-binding agents, such as nanoparticles (NPs).

### BACKGROUND

This section provides background information related to the present disclosure which is not necessarily prior art.

The quest to comprehensively analyze the blood plasma proteome has become crucial for advancing disease diagnosis and monitoring, as well as for biomarker discovery. Yet, obstacles such as identifying low-abundance proteins remain due to the prevalence of high-abundance proteins in plasma. The seven most abundant proteins collectively represent 85% of the total protein mass. Peptides from these high-abundance proteins, especially those of albumin, tend to dominate mass spectra, which impedes the detection of proteins with lower-abundance.

To address this challenge, techniques such as affinity depletion, Protein Equalizer™ technology, and electrolyte fractionation have been developed to reduce the concentration of these high-abundance proteins, thereby enhancing the detection of proteins with lower-abundance. Additionally, a range of techniques has been developed to enhance the throughput and depth of protein detection and identification, from advanced acquisition modes to methods that concentrate low-abundance proteins or peptides for liquid chromatography-mass spectrometry (LC-MS/MS) analysis. For instance, in the affinity depletion strategy, affinity chromatography columns are used with specific ligands that bind to high-abundance proteins, such as albumin, immunoglobulins, and haptoglobin. However, cost and labor associated with such depletion strategies hampers their application for large cohorts. As another example, the salting-out technique (also known as salt-induced precipitation) uses reagents (e.g., ammonium sulfate) to selectively precipitate high-abundance proteins, leaving the lower-abundance proteins in the supernatant. However, these methods can introduce biases in precipitating lower-abundance proteins as well, and therefore, additional robust strategies are needed to ensure low-abundance proteins with high diagnostic potential are not missed in biomarker discovery studies.

### SUMMARY

This section provides a general summary of the disclosure and is not a comprehensive disclosure of its full scope or all of its features.

In certain aspects, the present disclosure provides a method for detecting proteins and/or their proteoforms in a biological sample, such as human plasma. The present disclosure also provides a method for detecting one or more biomarker or pattern of one or more biomarker associated with a disease, such as a neoplastic disease or a neurological disease. Additionally, the present disclosure provides a method of diagnosing a disease in a subject.

In some embodiments, one or more small molecule may be added to a biological sample along with one or more protein-binding agent, such as a nanoparticle. In some embodiments, the method may include adding one or more small molecule to at least two biological samples, where each sample is from a different subject diagnosed with a disease. The protein-binding agent has a surface capable of binding proteins, allowing a protein corona to form on the surface of the protein-binding agent. Thus, a complex comprising the protein corona and the protein-binding agent may be generated. In some embodiments, the one or more small molecule may be a biomolecule, such as a lipid, a metabolite, a nutrient, or a plant-derived molecule. Further, in some embodiments the one or more small molecule may be a combination of different small molecules. In some embodiments, the small molecule may be phosphatidylcholine (PdtChos) or a derivative thereof.

In some embodiments, different proteins and/or their proteoforms in the protein corona may be detected. In some embodiments, one or more biomarker or pattern of one or more biomarker in the protein corona may be detected. In some embodiments, proteins and/or biomarkers may be detected, for example, by an antibody-based technique or a proteomics technique. In some embodiments, an increase in the number of proteins and/or their proteoforms detected in the protein corona may be observed, compared to a biological sample without adding the one or more protein-binding agent and one or more small molecule or compared to a biological sample with the one or more protein-binding agent added, but without the one or more small molecule added.

In further aspects, the present disclosure provides compositions including one or more small molecule, one or more protein-binding agent, and one or more biological sample. In some embodiments, the small molecule may be phosphatidylcholine. Additionally or alternatively, in some embodiments, the protein-binding agent may be nanoparticles.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described herein are for illustrative purposes only of selected embodiments and not all possible implementations and are not intended to limit the scope of the present disclosure.

FIG. 1 is an exemplary overview of an experimental process described herein. After exposing small molecules or small molecule combinations to human plasma, nanoparticles (NPs) were incubated with human plasma, purified and isolated, and used for analysis of the protein corona profile on the surface of the NPs using SDS-PAGE and LC-MS/MS.

FIG. 2 includes SDS-PAGE images analyzing protein corona coated NPs in the presence of eight individual small molecules and two small molecule combinations. The small molecules were analyzed at concentrations of 0, 10, 100, and 1000 μg/ml. The small molecule combinations include each of the four small molecules at the given concentration. For example, combination 1 at 10 μg/ml includes glucose at 10 μg/ml, triacylglycerols at 10 μg/ml, diacylglycerols at 10 μg/ml, and phosphatidylcholine at 10 μg/ml). Controls 1 and 2 correspond to SDS-PAGE results of all bare small molecules at 1,000 μg/ml in the absence of human plasma as follows: 1: glucose, 2: triacylglycerol, 3: diacylglycerol, 4: phosphatidylcholine, 5: phosphatidylethanolamine, 6: L-a-phosphatidylinositol, 7: inosine 5′-monophosphate, 8: vitamin B complex, 9: small molecule combination 1, and 10: small molecule combination 2.

FIG. 3A and FIG. 3B are bar graphs showing dynamic light scattering (DLS) (FIG. 3A) and zeta potential (FIG. 3B) analysis of bare NPs and untreated protein corona-coated NPs.

FIG. 4A-FIG. 4C are transmission electron microscope (TEM) images of NPs. FIG. 4A and FIG. 4B are TEM images of bare polystyrene NPs, while FIG. 4C is a TEM image of protein corona-coated NPs. The polydispersity index (PDI) of bare and protein corona-coated NPs were found to be 0.023 and 0.214, respectively.

FIG. 5 is a bar graph showing the number of quantified proteins in plasma, untreated protein corona and protein coronas in the presence of small molecules and small molecule combinations (mean ±SD of three technical replicates). The cumulative number of unique proteins identified across all conditions is also shown. For fair comparison, data analysis for each category (plasma, untreated protein corona, and protein corona in the presence of each small molecule or small molecule combination) was performed individually. Experiments were performed in three technical replicates and protein abundances were averaged for each condition.

FIG. 6 is a box and whisker plot showing the distribution of normalized intensities for proteins quantified in the plasma, untreated protein corona and protein coronas in the presence of small molecules and small molecule combinations (center line, median; box limits contain 50%; upper and lower quartiles, 75 and 25%; maximum, greatest value excluding outliers; minimum, least value excluding outliers; outliers, more than 1.5 times of upper and lower quartiles). Experiments were performed in three technical replicates and protein abundances were averaged for each condition.

FIG. 7 is a heat map showing the hierarchical clustering of all proteins (1793 cumulative proteins) quantified across all samples (plasma, untreated protein corona and protein coronas in the presence of small molecules and small molecule combinations). Experiments were performed in three technical replicates and protein abundances were averaged for each condition.

FIG. 8 is a heat map showing the clustering of 117 shared proteins across all samples (plasma, untreated protein corona and protein coronas in the presence of small molecules and small molecule combinations). Experiments were performed in three technical replicates and protein abundances were averaged for each condition.

FIG. 9 is a heat map showing the correlation of plasma proteome profiles with a Pearson correlation of the 117 shared proteins across all the samples (plasma, untreated protein corona and protein coronas in the presence of small molecules and small molecule combinations) at 10-1000 μg/ml.

FIG. 10 includes two charts showing that different small molecule combinations can enrich or deplete specific proteins. The charts show the number of unique proteins that were quantified in a given group which were not quantified in the plasma or with bare NPs.

FIG. 11A-FIG. 11D show the enriched and depleted proteins for small molecule combinations 1 (FIG. 11A and FIGS. 11B) and 2 (FIG. 11C and FIG. 11D) in comparison to the untreated protein corona. Respective pathway analysis was performed for all the depleted and enriched proteins (FIG. 11B and FIG. 11D). Significance was calculated using unequal variance (the Welch Two Sample t-test).

FIG. 12 includes scatter plots showing the enrichment and depletion of specific proteins (only those shared) by spiking small molecules in NP protein corona vs. the abundance of proteins in the untreated NP protein corona. Only the results for the highest concentration of each small molecule (1000 μg/ml) are shown.

FIG. 13 includes bar graphs showing pathway enrichment in KEGG and biological processes for all significantly enriched and depleted proteins for each small molecule cumulatively across all concentrations.

FIG. 14 includes a combined enrichment plot for KEGG and biological processes for all small molecules and small molecule combinations vs. untreated protein corona, cumulatively across all concentrations.

FIG. 15 includes bar graphs showing the classification of quantified protein corona of various small molecules and small molecule combinations according to their physiological functions.

FIG. 16 is a graph representing the impact of spiking small molecules or small molecule combinations on the proteome dynamic range. The dynamic range (order of magnitude) of the proteomics analysis for different samples is shown.

FIG. 17A-FIG. 17C are graphs showing the comparative analysis of protein (albumin —FIG. 17A, serotransferrin—FIG. 17B, and haptoglobin—FIG. 17C) abundance and rankings in untreated plasma versus protein corona profiles, both with and without the addition of small molecules or small molecule combinations.

FIG. 18A-FIG. 18C include charts showing protein abundance following incubation with NPs and phosphatidylcholine (PdtChos). FIG. 18A shows a stream (or alluvial) diagram illustrating the significant depletion of abundant plasma proteins, particularly albumin, following the incubation of plasma with NPs and PtdChos (only shared proteins with plasma are included). FIG. 18B shows the total count of proteins identified in plasma samples incubated with NPs, comparing treatments with and without PtdChos at various concentrations (mean ±SD of three technical replicates). FIG. 18C shows a stream diagram demonstrating the depletion pattern of abundant plasma proteins, especially albumin, in response to NP addition and enhanced with escalating concentrations of PtdChos (only shared proteins with plasma are included).

### DETAILED DESCRIPTION

### A. Introduction

Recently, nanoparticles (NPs) have gained attention for their ability to enhance biomarker discovery through analysis of the spontaneously-forming protein/biomolecular corona (i.e., a layer of biomolecules, primarily proteins, that forms on the surface of NPs when exposed to plasma or other biological fluids). The NP protein corona can contain a unique ability to concentrate proteins with lower abundance, easily reducing the proteome complexity for LC-MS/MS analysis.

Application of single NPs for biomarker discovery has limitations in achieving deep proteome coverage, typically enabling the detection of only hundreds of proteins. To enhance proteome coverage and quantify a higher number of plasma proteins, the use of a protein corona sensor array or multiple NPs with distinct physicochemical properties can be implemented. This approach leverages the unique protein corona that forms on each NP to increase proteome coverage but carries the drawback of having to analyze multiple NP samples and needing to test many NP types to increase proteome coverage.

Although protein coronas are known to form on the surface of NPs when exposed to biological fluids, the present disclosure provides a new way to detect low-abundance proteins via the addition of small molecules. The addition of small molecules, or a combination of small molecules, to the biological fluid along with a protein-binding agent having a surface, such as NPs, can generate distinct protein corona patterns and significantly expand the dynamic range of the plasma proteome that can be captured and detected by LC-MS/MS. The inventors unexpectedly found that the addition of small molecules, such as phosphatidylcholine (PtdChos), can lead to a substantial increase in proteome coverage. Thus, this approach may seamlessly integrate with existing LC-MS/MS workflows to further enhance the depth of plasma proteome analysis for biomarker discovery.

The disclosed methods may also allow for early detection of health spectrum conditions, such as diseases or disorders, which can prevent or delay problems from the disease or condition and may improve outcomes for a patient, such as extending a patient's life and/or quality of life. For example, the disclosed methods may help prevent or reduce the risk of developing conditions such as obesity, heart disease, liver disease, kidney disease, depression, cancer, etc. As various types of cancers can change the composition of blood plasma, even in their early stages, one approach for early detection is molecular blood analysis for one or more biomarkers. The present disclosure provides a novel approach to improve the ability to detect cancer, for example at early stages.

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

### B. Definitions

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this disclosure, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art. Particularly in reference to a given quantity, number or percentage, “about” is meant to encompass deviations of plus or minus ten percent (±10). For example, about 5% encompasses any value between 4.5% to 5.5%, such as 4.5, 4.6, 4.7, 4.8, 4.9, 5, 4.1, 5.2, 5.3, 5.4, or 5.5. Accordingly, unless otherwise indicated, the numerical parameters set forth in this specification and attached claims are approximations that can vary depending upon the desired properties sought to be obtained by the presently disclosed subject matter.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As will be understood by one skilled in the art, for any and all purpose, all ranges disclosed herein also encompass any and all possible subranges and combinations of subranges thereof. Furthermore, as will be understood by one skilled in the art, a range includes each individual member.

Unless defined otherwise, technical and scientific terms used herein have the same meaning as commonly understood by a person of ordinary skill in the art. In particular, this disclosure utilizes routine techniques in the field of wet granulation and pectin-comprising substances.

“Protein corona”, often referred to as “PC”, as used herein is a biomolecular shell that forms on the surface of nanoparticles (NPs) during their interactions with biological fluids, which changes over time. The term “biomolecule corona” refers to multiple different biomolecules that are able to bind to a protein-binding agent having a surface, such as a nanoparticle. The term “biomolecule corona” encompasses “protein corona” which is a term used in the art to refer to the proteins, lipids and other plasma components that bind a protein-binding agent, such as nanoparticles when they come into contact with biological fluids. For use herein, the term “protein corona” encompasses both the soft and hard protein corona as referred to in the art, see, e.g., Milani, et al., “Reversible versus Irreversible Binding of Transferrin to Polystyrene Nanoparticles: Soft and Hard Corona,” ACS NANO, 2012, 6(3), pp. 2532-2541; Mirshafiee, et al., “Impact of protein pre-coating on the protein corona composition and nanoparticle cellular uptake,” Biomaterials, vol. 75, January 2016 pp. 295-304, Mahmoudi, et al., “Emerging understanding of the protein corona at the nano-bio interfaces,” Nanotoday, 11(6) December 2016, pp. 817-832, and Mahmoudi, et al., “Protein-Nanoparticle Interactions: Opportunities and Challenges,” Chem. Rev., 2011, 111(9), pp. 5610-5637, the contents of which are incorporated by reference in their entireties. As described in the art, adsorption curve shows the build-up of a strongly bound monolayer up to the point of monolayer saturation (at a geometrically defined protein-to-nanoparticle ratio), beyond which a secondary, weakly bound layer is formed. While the first layer is irreversibly bound (hard corona), the secondary layer (soft corona) exhibits dynamic exchange. Proteins that adsorb with high affinity form what is known as the “hard” corona, consisting of tightly bound proteins that do not readily desorb, and proteins that adsorb with low affinity form the “soft” corona, consisting of loosely bound proteins. Soft and hard corona can also be defined based on their exchange times. Hard corona usually shows much larger exchange times in the order of several hours. See, e.g., M. Rahman, et al., Protein-Nanoparticle Interactions, Spring Series in Biophysics 15, 2013, incorporated by reference in its entirety.

A “protein-binding agent” as used herein is any agent that binds, interacts with, or attracts one or more protein in a biological sample, and has a surface for protein binding. In some embodiments, the protein-binding agent may be referred to as a protein-interacting agent, a protein-attracting agent, and/or a protein corona-forming agent. In some embodiments, the protein-binding agent may be a nanoscale material and/or a microscale material.

A “nanoscale material” or “nanomaterial” as used herein is a material of which a single unit is sized (in at least one dimension) between 1 nanometer (nm) and 999 nm. A non-limiting list of nanomaterials includes nanoparticles, nanorods, nanospheres, nanodisks, nanoclusters, nanofibers, and nanotubes.

A “microscale material” or “micromaterial” as used herein is a material of which a single unit is sized (in at least one dimension) between 1000 nm and 100,000 nm. A non-limiting list of micromaterials includes microparticles, microrods, microspheres, and microbeads.

Examples of suitable nanoscale and/or microscale materials include, but are not limited to, organic materials, non-organic materials, or combinations thereof. In some embodiments, the materials are micelles, liposomes, iron oxide, graphene, silica, protein-based materials, polystyrene, silver, and gold materials,, such as colloidal gold, quantum dots, palladium, platinum, titanium, and combinations thereof. In some embodiments, nanoparticles are liposomes. One skilled in the art is able to select and prepare suitable nanoscale and/or microscale material(s).

A “small molecule” as used herein is a molecule having a molecular weight of less than 5 kilodaltons (kDa). For the purposes of this disclosure, molecular weight may be calculated by the following formula: Molecular weight (in the dalton unit (Da) or the unified atomic mass unit (u))=Σ((atomic mass of element)ₙ×(#of atoms of that element)ₙ). The small molecule can be synthetic or natural. In other words, the small molecule may be synthetically generated or it can be naturally produced. The small molecules described in the methods herein may also be referred to as protein-recruitment agents because they encourage recruitment of different proteins (and other types of biomolecules) to the surface of materials, such as nanoparticles. In some embodiments, the small molecule may also be referred to as a “high-abundance protein-binding agent” or a “high-abundance protein-interacting agent.”

“High-abundance protein” as used herein refers to one of the seven most abundant proteins (albumin, IgG, antitrypsin, IgA, transferrin, haptoglobin, and fibrinogen) in human plasma. Such proteins collectively represent 85% of the total protein mass in human plasma.

“Low-abundance protein” as used herein refers to any proteins present in human plasma that are not included in the seven high-abundance proteins.

A “biomolecule” as used herein is a molecule produced by a living organism and essential to one or more biological processes. A biomolecule may also be synthetically generated to mimic the function of a naturally generated biomolecule. Examples of suitable biomolecules include, but are not limited to, macromolecules (such as proteins, carbohydrates, lipids, nucleic acids, etc.) as well as smaller molecules (such as vitamins, hormones, etc.). A biomolecule may also be referred to herein as a biological material. Thus, a small molecule as used herein can be considered a “small biomolecule” if it fits the definition of a small molecule (a molecule having a molecular weight of less than 5 kilodaltons (kDa)) and the definition of a biomolecule (a molecule produced by a living organism and essential to one or more biological processes or a molecule synthetically generated to mimic the function of a naturally generated biomolecule).

A “metabolite” as used herein is an intermediate or end product of metabolism. It is intended to include all natural, synthetic, and biological small molecules, such as amino acids, alcohols, polyols, alkaloids, organic acids, sugars (e.g., glucose) as well as nucleotides (e.g., inosine-5′-monophosphate and guanosine-5′-monophosphate).

“Lipids” as used herein are a group of organic compounds including all natural, synthetic, and biological fatty compounds, such as glycerolipids (e.g., triacylglycerols also known as triglycerides, TG or TAG; diacylglycerols also known as diglycerides, DG or DAG, such as 1,2-diacylglycerols and 1,3-diacylglycerols), glycerophospholipids (e.g., phosphatidylcholine, phosphatidylethanolamine, L-α-phosphatidylinositol), sterol lipids, very-low-density lipoprotein (VLDL), low density lipoprotein (LDL), and high-density lipoprotein (HDL), fatty acids, prenol lipids, and sphingolipids.

“Nutrient” as used herein is a substance used by an organism to survive, grow, and reproduce. In some cases a metabolite can also be considered a nutrient, such as amino acids, fatty acids, vitamins (e.g. vitamin B complex), minerals and choline.

“Plant-derived molecule” as used herein refers to any molecule derived from a plant. Suitable plant-derived molecules include small molecules such as auxin, gibberellic acid, alkaloids, phenylpropanoids; plant-made biologics, such as anti-cancer biologics; and phytopharmaceutical drugs, such as those with properties against human health problems such as allergy, inflammation, etc.

The term “endogenous” as used herein refers to a substance, e.g. a nucleic acid, protein, enzyme, small molecule, etc., that is produced from within a host organism (e.g., a human) and/or that is naturally occurring or naturally found inside a host organism. Thus, an endogenous substance refers to a substance produced by and found inside a host organism. In some embodiments, an endogenous substance may be encoded by the genome of the host organism. In some embodiments, the substance may be encoded by an autonomously replicating plasmid carried by the host organism. In some embodiments, an endogenous substance is a substance that was present in a host organism's biological sample when the biological sample was originally isolated from nature, i.e., the substance is native to the organism. For example, an “endogenously produced” substance may be expressed/generated by a host organism's own machinery. In other words, the host organism has not been genetically engineered to produce the substance. In other words, a host organism may endogenously produce a native or non-native substance.

In contrast, an “exogenous” substance, e.g., a nucleic acid, protein, enzyme, small molecule, etc., as used herein, refers to a substance that is not encoded by or produced by the host organism (human or non-human, such as a mouse, rat, pig, etc.), and which is therefore added to the host organism or biological sample taken from the host organism from outside of the host organism. For example, “exogenously added” may refer to adding a substance, such as a small molecule, to the host organism or a biological sample taken from the host organism. A nucleic acid sequence encoding a variant (i.e., mutant) polypeptide, when added to a host organism or host organism cell, is one example of an exogenous nucleic acid sequence. The exogenous nucleic acid sequence can encode a polypeptide or an enzyme that is also otherwise endogenous or native to the cell. Such an encoded polypeptide or enzyme can be considered “exogenously expressed.” For example, to achieve overexpression of an endogenous gene, additional copies of the gene can be introduced into the cell (e.g., in a vector, such as a plasmid). Such additional copies of the endogenous gene can be considered as “exogenous” (e.g., exogenous gene(s) or an exogenous nucleic acid sequence(s)), because the additional copies are introduced into the cell from outside the cell. An “exogenous gene” or “exogenous nucleic acid sequence” also refers to a native (or endogenous) gene or nucleic acid sequence that is deregulated (e.g., upregulated, downregulated, or attenuated) or otherwise altered or modified, for example, by operably linking it to a regulatory element. An exogenous nucleic acid sequence or exogenous gene can also be used to express or overexpress a heterologous polypeptide or enzyme in a cell. Thus, an exogenous nucleic acid sequence or an exogenous gene can encode a polypeptide (e.g., an enzyme) that is native to the cell, that is otherwise endogenous to the cell, or that is heterologous to the cell.

As used herein the term “native” refers to the form of a composition, such as a small molecule, that is isolated from nature, or to a composition that is in its natural state without intentionally introduced mutations in the structural sequence and/or without any engineered changes in expression such as e.g., changing a developmentally regulated gene to a constitutively expressed gene. As used herein, “native” also refers to “wildtype” or “wild-type,” in which the composition is present in both sequence, quantity, and relative quantity as typically found in the organism as naturally found. Wild-type organisms may serve as a control and/or reference for determination of cellular functions. A native molecule, e.g. a lipid, gene, nucleic acid sequence, polypeptide, or enzyme, for example, is typically endogenous to a cell, i.e., found in or produced by the cell. An exogenous nucleic acid sequence or an exogenous gene can encode a native polypeptide or enzyme, for example, where additional copies of a native gene or nucleic acid sequence are added to the cell from outside the cell, or where a native gene or nucleic acid sequence is deregulated or altered, e.g., by operably coupling it to a regulatory element that is not native or endogenous to the cell.

The term “non-native” is used herein to refer to nucleic acid sequences, amino acid sequences, polypeptide sequences, enzymes, and/or small molecules that do not occur naturally in the host. Heterologous genes and polypeptides are considered “non-native.” A nucleic acid sequence or amino acid sequence that has been removed from a host cell, subjected to laboratory manipulation, and introduced or reintroduced into a host cell, is also considered “non-native.” Synthetic or partially synthetic genes introduced into a host cell are “non-native.” Non-native genes further include genes that are endogenous and/or native to the host microorganism but that are operably linked to one or more heterologous regulatory sequences that have been recombined into the host genome. A naturally occurring gene under the control of a heterologous regulatory sequence is considered “non-native.” In some embodiments, an organism comprising a non-native gene may be utilized as a control and/or reference for an organism having additional and/or different variations from wild-type organisms.

“Sample” as used herein refers to a biological sample or a complex biological sample obtained from a subject. Suitable biological samples include, but are not limited to, biological fluids, such as systemic blood, plasma, serum, lung lavage, cell lysates, menstrual blood, urine, processed tissue samples, amniotic fluid, cerebrospinal fluid, tears, saliva, semen and the like. In a particular embodiment, the sample is a whole blood, plasma or serum sample.

“Health spectrum” as used herein covers a broad range of health states, including complete well-being, minor health issues, chronic conditions, and severe illnesses. The health spectrum adopts a holistic and dynamic view of health, emphasizing prevention and the continuum of health states. The health spectrum is concerned with overall well-being and the factors that influence it, whether they lead to optimal health or contribute to illness. The health spectrum emphasizes preventive care, lifestyle modifications, and overall health maintenance. Whereas a “disease/disorder” focuses on specific pathological conditions affecting health. A disease/disorder adopts a more specific, diagnostic approach, focusing on identifying and treating particular conditions. A disease/disorder is focused on the pathological aspects of health, identifying specific illnesses or dysfunctions to address them effectively. Disease/disorder emphasizes medical intervention, treatment plans, and symptom management for specific conditions.

A disease/disorder is one example of a health spectrum condition. Other conditions include pre-diagnosis states where one may be at risk of developing a disease or disorder, but has not reached the level of a diagnosed disease yet.

### C. Methods

Method for detecting proteins and other biomolecules in a biological sample

### General Methods

In one embodiment, the present disclosure provides a method for detecting biomolecules, such as proteins and/or their proteoforms, in a biological sample, including adding one or more small molecule and one or more protein-binding agent to the biological sample. The protein-binding agent has a surface capable of binding proteins and allowing a protein corona to form on the surface of the protein-binding agent, thus forming a complex including the protein corona and the protein-binding agent. The number of proteins and/or their proteoforms in the protein corona may then be detected using techniques such as antibody-based techniques or proteomics techniques.

The one or more small molecules and one or more protein-binding agent may be added to the biological sample in any order. For example, in some embodiments, the one or more small molecule may be added to the biological sample, then the one or more protein-binding agent may be added to the biological sample containing the one or more small molecule. Alternatively, in some embodiments, the one or more protein-binding agent may be added to the biological sample, then the one or more small molecule may be added to the biological sample containing the one or more protein-binding agent. Alternatively, in some embodiments, the one or more small molecule and one or more protein-binding agent may be added to the biological sample at or approximately at the same time.

After addition of the one or more small molecule and/or the protein-binding agent to the biological sample, the sample containing the one or more small molecule and/or the one or more protein-binding agent may be incubated to allow a protein corona to form on the surface of the protein-binding agent. For example, in some embodiments, the biological sample may be incubated with the one or more small molecule. In some embodiments, the sample may be incubated with the one or more protein-binding agent. In some embodiments, the sample may be incubated with the one or more small molecule and the one or more protein-binding agent. In some embodiments, the biological sample may be incubated for at least 10 seconds to about 24 hours. For example, the biological sample may be incubated for at least about 10 seconds, at least about 15 seconds, at least about 20 seconds, at least about 25 seconds, at least about 30 seconds, at least about 40 seconds, at least about 50 seconds, at least about 60 seconds, at least about 90 seconds, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 45 minutes, at least about 50 minutes, at least about 60 minutes, at least about 90 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, and include any time and increment in between. In a specific embodiment, the biological sample may be incubated for about 1 hour. In some embodiments, the biological sample may be incubated more than once, for example, the biological sample may be incubated one, two, three, four, or five times.

The incubation temperature can be determined by one skilled in the art, and includes temperatures between about 4° C. to about 40° C., about 4° C. to about 20° C., about 10° C. to about 15° C., about 10° C. to about 40° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 25°C., about 30° C., about 35°, about 37° C., etc. In some embodiments, the method may be performed at room temperature (e.g., about 37° C. ; e.g., about 35° C. to about 40° C.).

In some embodiments, the biological sample may be diluted. In some embodiments, the biological sample may be diluted using a suitable buffer, such as phosphate buffer saline (PBS), Tris-HCl buffer, ammonium bicarbonate buffer, HEPES (4-(2-hydroxyethyl)-1-piperazineethanesulfonic acid) buffer, MOPS (3-(N-morpholino)propanesulfonic acid) buffer, PIPES (1,4-Piperazinediethanesulfonic acid) buffer, or EPPS (4-(2-Hydroxyethyl)-1-piperazinepropanesulfonic acid) buffer. In a specific embodiment, the biological sample may be diluted using PBS. For example, the biological sample may be diluted to a final concentration of about 25% to about 85%, about 30% to about 80%, about 35% to about 75%, about 40% to about 70%, about 45% to about 65%, about 50% to about 60%, about 25%, about 30%, about 35%, about 40%, about 45%, about 50%, about 55%, about 60%, about 65%, about 70%, about 75%, about 80%, or about 85%. In a specific embodiment, the biological sample may be diluted to a final concentration of about 55% using PBS.

### Biological Sample

In some embodiments, a biological sample may include, but is not limited to, biological fluids, such as systemic (whole) blood, plasma, serum, lung lavage, cell lysates, menstrual blood, urine, processed tissue samples, amniotic fluid, cerebrospinal fluid, tears, saliva, semen and the like. In a particular embodiment, the biological sample may be a whole blood, plasma, or serum sample. In another particular embodiment, the biological sample may be a plasma sample. In some embodiments, the biological sample may include more than one biological sample. For example, the biological sample may include 1, 2, 3, 4, 5, 6, 7, 8, 9, 10, 15, 20, 25, 30, or more of the same or different biological samples.

In some embodiments, biological fluids or complex biological samples may be prepared by methods and kits known in the art. For example, some biological samples (e.g. menstrual blood, blood samples, semen, etc.) may first be centrifuged at low speed to remove cell debris, blood clots and other cellular components that may interfere with the methods described herein. In other embodiments, for example, tissue specimens may be processed, e.g. tissue samples may be minced or homogenized, treated with enzymes to break up the tissue and/or centrifuged to remove cellular debris allowing for the assaying and extraction of molecules within the tissue samples. Suitable methods of isolating and/or properly preparing and storing blood samples are known in the art, and may include, but are not limited to, the addition of an anti-coagulant agent.

In some embodiments, the method for detecting proteins in a biological sample may include a step for depleting one or more proteins in a biological sample. For example, depleting one or more proteins in a biological sample may include running the biological sample through a depletion column or through a spin column with resin. In some embodiments, running the biological sample through a depletion column or spin column with resin may reduce the complexity of biological samples for analysis via antibody-based techniques or proteomics techniques. For example, the complexity of biological samples may be reduced for top-down, middle-down, and bottom-up proteomics analysis. In some embodiments, depletion columns or spin columns may be used to reduce the complexity of biological samples such as serum, plasma, etc., which contain high concentrations of albumin and immunoglobulins. For example, depletion columns or spin columns may be used to remove highly abundant proteins such as albumin and IgG from biological samples. In some embodiments, a suitable depletion or spin column may be a High Select™ Depletion Spin Column (Thermo Scientific™). In some embodiments, protein depletion methods may be used for applications in drug delivery and/or imaging. In some embodiments, protein depletion methods, such as those including depletion or spin columns, may include particles, such as lipid-based NPs, with a small molecule, such as PtdChos, on their surface. Thus, in some embodiments, proteins, such as albumin, may be attracted and/or bound to the surface of the small molecule-coated NPs, which may enhance the small molecule-coated NP's blood circulation time and/or allow them to be removed quickly by the immune system.

Biomolecule, e.g. Protein, Detection

The method described herein may be used to detect biomolecules in a biological sample. Biomolecules that may be detected by the disclosed method may include small molecules (such as lipids, fatty acids, glycolipids, sterols, monosaccharides, vitamins, hormones, neurotransmitters, metabolites, etc.), monomers (such as amino acids, monosaccharides, isoprene, nucleotides, etc.) oligomers (such as oligopeptides, oligosaccharides, terpenes, oligonucleotides, etc.), and polymers (such as polypeptides, proteins and/or their proteoforms, polysaccharides, polyterpenes, polynucleotides, nucleic acids, etc.). In some embodiments, the method described herein may be used to detect proteins and/or their proteoforms. In some embodiments, proteoforms of proteins may be different forms of a protein produced from the genome with a variety of biological variations, such as sequence variations, splice isoforms, post-translational modifications, etc., which may alter the primary sequence and composition at the whole-protein level. In some embodiments, proteoforms may carry different biological functions.

In some embodiments, the method described herein may be used to detect a number of unique biomolecules in a biological sample. For example, the method may be used to detect a number of different proteins and/or their proteoforms in a biological sample. Thus, the method described herein may be used to detect one type of protein and/or its proteoforms and/or the method may be used to detect more than one type of protein and/or their proteoforms. In some embodiments, the method may be used to detect an amount of one type of protein present in a biological sample and/or present in a protein corona. In some embodiments, the method may be used to detect an amount of more than one type of protein present in a biological sample and/or present in a protein corona. In some embodiments, the method may be used to detect how many distinct proteins are present in a biological sample and/or are present in a protein corona.

### Small Molecules

The method described herein uses a combination of small molecules and protein-binding agents to detect biomolecules, such as proteins, in a biological sample. As described herein, “one or more small molecule” covers a “small molecule combination” (i.e., a combination of one or more small molecules). In some embodiments, the method includes adding one or more small molecule or small molecule combination to the biological sample. In some embodiments, the method may include adding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc. small molecules to the biological sample. In some embodiments, the method may include adding a combination of one or more small molecules to the biological sample. In some embodiments, the small molecule combination may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc. different small molecule types. In some embodiments, the method may include adding one or more small molecule combination to the biological sample. In some embodiments, the method may include adding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc. small molecule combinations to the biological sample. In some embodiments, the number of small molecules or small molecule combinations added to the biological sample may be any number to reach a desired concentration of small molecules or small molecule combinations in the biological sample.

In some embodiments, the small molecule or small molecule combination may be added to the biological sample at a concentration of about 1 pg/ml to about 1 g/ml. For example, in some embodiments, the small molecule or small molecule combination may be added to the biological sample at a concentration of about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1,000 μg/ml, about 2,000 μg/ml, about 3,000 μg/ml, about 4,000 μg/ml, about 5,000 μg/ml, about 6,000 μg/ml, about 7,000 μg/ml, about 8,000 μg/ml, about 9,000 μg/ml, about 10,000 μg/ml, about 11,000 μg/ml, about 12,000 μg/ml, about 13,000 μg/ml, about 14,000 μg/ml, about 15,000 μg/ml, about 16,000 μg/ml, about 17,000 μg/ml, about 18,000 μg/ml, about 19,000 μg/ml, about 20,000 μg/ml, about 10 μg/ml, to about 10,000 μg/ml, about 20 μg/ml, to about 10,000 μg/ml, about 30 μg/ml to about 10,000 μg/ml, about 40 μg/ml to about 10,000 μg/ml, about 50 μg/ml, to about 10,000 μg/ml, about 60 μg/ml to about 10,000 μg/ml, about 70 μg/ml to about 10,000 μg/ml, about 80 μg/ml to about 10,000 μg/ml, about 90 μg/ml to about 10,000 μg/ml, about 100 μg/ml, to about 10,000 μg/ml, about 200 μg/ml to about 9,000 μg/ml, about 300 μg/ml to about 8,000 μg/ml, about 400 μg/ml to about 7,000 μg/ml, about 500 μg/ml to about 6,000 μg/ml, about 600 μg/ml to about 5,000 μg/ml, about 700 μg/ml to about 4,000 μg/ml, about 800 μg/ml to about 3,000 μg/ml, about 900 μg/ml to about 2,000 μg/ml, about 950 μg/ml to about 1,500 μg/ml, about 10 μg/ml to about 1,000 μg/ml, about 20 μg/ml to about 1,000 μg/ml, about 30 μg/ml to about 1,000 μg/ml, about 40 μg/ml to about 1,000 μg/ml, about 50 μg/ml to about 1,000 μg/ml, about 60 μg/ml to about 1,000 μg/ml, about 70 μg/ml to about 1,000 μg/ml, about 80 μg/ml to about 1,000 μg/ml, about 90 μg/ml to about 1,000 μg/ml, about 10 μg/ml to about 900 μg/ml, about 10 μg/ml to about 800 μg/ml, about 10 μg/ml to about 700 μg/ml, about 10 μg/ml to about 600 μg/ml, about 10 μg/ml to about 500 μg/ml, about 10 μg/ml to about 400 μg/ml, about 10 μg/ml to about 300 μg/ml, about 10 μg/ml to about 200 μg/ml, about 10 μg/ml to about 100 μg/ml, about 10 μg/ml to about 50 μg/ml, about 1,000 μg/ml to about 20,000 μg/ml, about 2,000 μg/ml to about 19,000 μg/ml, about 3,000 μg/ml to about 18,000 μg/ml, about 4,000 μg/ml to about 17,000 μg/ml, about 5,000 μg/ml to about 16,000μg/ml, about 6,000 μg/ml to about 15,000 μg/ml, about 7,000 μg/ml to about 14,000 μg/ml, about 8,000 μg/ml to about 13,000 μg/ml, about 9,000 μg/ml to about 12,000 μg/ml, about 9,000 μg/ml to about 11,000 μg/ml, or about 9,500 μg/ml to about 10,500 μg/ml. In some embodiments, about 10 μg/ml of a small molecule or small molecule combination may be added to the biological sample. In some embodiments, about 100 μg/ml of a small molecule or small molecule combination may be added to the biological sample. In some embodiments, about 1,000 μg/ml of a small molecule or small molecule combination may be added to the biological sample. In some embodiments, one or more small molecule or small molecule combination may be added to the biological sample at diverse concentrations. In some embodiments, the diverse concentrations may be from about 1 pg/ml to about 1 g/ml.

In some embodiments, a small molecule may have a molecular weight of less than about 5 kDa, about 4.9 kDa, about 4.8 kDa, about 4.7 kDa, about 4.6 kDa, about 4.5 kDa, about 4.4 kDa, about 4.3 kDa, about 4.2 kDa, about 4.1 kDa, about 4.0 kDa, about 3.9 kDa, about 3.8 kDa, about 3.7 kDa, about 3.6 kDa, about 3.5 kDa, about 3.4 kDa, about 3.3 kDa, about 3.2 kDa, about 3.1 kDa, about 3.0 kDa, about 2.9 kDa, about 2.8 kDa, about 2.7 kDa, about 2.6 kDa, about 2.5 kDa, about 2.4 kDa, about 2.3 kDa, about 2.2 kDa, about 2.1 kDa, about 2.0, about 1.9, about 1.8, about 1.7, about 1.6, about 1.5, about 1.4, about 1.3, about 1.2, about 1.1 kDa, about 1.0 kDa, about 0.9 kDa, about 0.8 kDa, about 0.7 kDa, about 0.6 kDa, about 0.5 kDa, about 0.4 kDa, about 0.3 kDa, about 0.2 kDa, or about 0.1 kDa. In other words, a small molecule may be any molecule that weighs less than about 5 kDa.

A small molecule as used herein may be considered a “small biomolecule” if it is a molecule having a molecular weight of less than 5 kilodaltons (kDa) and is produced by a living organism and essential to one or more biological processes (or is synthetically generated to mimic the function of a naturally generated biomolecule). In some embodiments, the small molecule may be synthetically generated (i.e., artificial). Alternatively, in some embodiments, the small molecule may be naturally produced. In some embodiments, the small molecule may be endogenous to the host organism from which the biological sample is taken. In other embodiments, the small molecule may be exogenous to the host organism from which the biological sample is taken. Additionally or alternatively, the small molecule may be native or non-native to the host organism. For example, the small molecule may be native or non-native to the host organism from which the biological sample is taken, e.g. a lipid, protein, or nucleic acid, and exogenously added to the biological sample.

In some embodiments, the small molecule may encourage recruitment of different proteins (and other types of biomolecules) to the surface of protein-binding agents, such as nanoparticles. In some embodiments, the small molecule may be capable of altering a protein corona. For example, the small molecule may be capable of altering a protein corona that has formed on the surface of a protein-binding agent, such as a nanoparticle. In some embodiments, the small molecule may be capable of depleting high-abundance plasma proteins such as albumin, IgG, antitrypsin, IgA, transferrin, haptoglobin, and fibrinogen in a biological sample. Thus, in some embodiments, the small molecule may be referred to as a “high-abundance protein depleting agent”. In some embodiments, the small molecule may be capable of depleting at least one, at least two, at least three, at least four, at least five, at least six, or all seven of the high-abundance plasma proteins. In a specific embodiment, the small molecule may be capable of depleting albumin.

In some embodiments, a small molecule may physically or chemically interact with one or more proteins in a biological sample. Thus, in some embodiments, the small molecule may be referred to as a “protein-interacting agent”. In some embodiments, the small molecule may bind proteins in the biological sample. In some embodiments, the small molecule may bind albumin, IgG, antitrypsin, IgA, transferrin, haptoglobin, and/or fibrinogen. In specific embodiments, the small molecule may bind one or more abundant protein types. Thus, in some embodiments, binding the abundant proteins may deplete the number of abundant proteins in a biological sample. In some embodiments, a small molecule may change the conformation of one or more proteins in the biological sample.

In some embodiments, the small molecule may be a metabolite and/or a derivative thereof. In some embodiments, a metabolite may be any natural, synthetic, or biological small molecule, such as an amino acid, alcohols, polyols, alkaloids, organic acids, sugars (e.g., glucose) as well as nucleotides (e.g., inosine-5′-monophosphate and guanosine-5′-monophosphate).

In some embodiments, the small molecule may be a lipid and/or a derivative thereof. In some embodiments, lipids may be a group of organic compounds including all natural, synthetic, and biological fatty compounds, such as glycerolipids (e.g., triacylglycerols also known as triglycerides, TG or TAG; diacylglycerols also known as diglycerides, DG or DAG, such as 1,2-diacylglycerols and 1,3-diacylglycerols), glycerophospholipids (e.g. phosphatidylcholine, phosphatidylethanolamine, L-α-phosphatidylinositol), sterol lipids, very-low-density lipoprotein (VLDL), low density lipoprotein (LDL), and high-density lipoprotein (HDL), fatty acids, prenol lipids, and sphingolipids.

In some embodiments, the small molecule may be a nutrient and/or a derivative thereof. In some embodiments, a metabolite can also be considered a nutrient, such as amino acids, fatty acids, vitamins (e.g. vitamin B complex), minerals and choline.

In some embodiments, the small molecule may be a plant-derived molecule and/or a derivative thereof, such as a small molecule (e.g., auxin, gibberellic acid, alkaloid, phenylpropanoid), a plant-made biologic (e.g., anti-cancer biologics), or phytopharmaceutical drugs (e.g., those with properties against human health problems such as allergy, inflammation, etc.).

In some embodiments, the small molecule may be a metabolite and/or a derivative thereof, lipid and/or a derivative thereof, nutrient and/or a derivative thereof, plant-derived molecule and/or a derivative thereof, or a combination thereof.

In some embodiments, the small molecule may be a triacylglycerol and/or a derivative thereof, diacylglycerol, such as 1,2 and 1,3 diacylglycerol, and/or a derivative thereof, glycerophospholipid and/or a derivative thereof, glucose and/or a derivative thereof, inosine 5′-monophosphate and/or a derivative thereof, vitamin B complex and/or a derivative thereof, phosphatidylcholine and/or a derivative thereof, phosphatidylethanolamine and/or a derivative thereof, phosphatidylserine and/or a derivative thereof, phosphatidic acid and/or a derivative thereof, phosphatidylinositol and/or a derivative thereof, phosphatidylglycerol and/or a derivative thereof, cardiolipin and/or a derivative thereof, L-α-phosphatidylinositol and/or a derivative thereof, or a combination thereof.

Combinations of small molecules are also contemplated for use herein. For example, in some embodiments, the small molecules may be a combination of a triacylglycerol and/or a derivative thereof, a diacylglycerol and/or a derivative thereof, and a glycerophospholipid and/or a derivative thereof. Additionally or alternatively, the small molecules may be a combination of glucose and/or a derivative thereof, inosine 5′-monophosphate and/or a derivative thereof, and vitamin B complex and/or a derivative thereof. Additionally or alternatively, the small molecules may be a combination of a triacylglycerol and/or a derivative thereof, a diacylglycerol and/or a derivative thereof; a glycerophospholipid and/or derivative thereof, glucose and/or a derivative thereof, inosine 5′-monophosphate and/or a derivative thereof, and vitamin B complex and/or a derivative thereof Additionally or alternatively, the small molecules may be a combination of glucose and/or a derivative thereof, a triacylglycerol and/or a derivative thereof, a diacylglycerol and/or a derivative thereof; and phosphatidylcholine and/or a derivative thereof. Additionally or alternatively, the small molecules may be a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof.

### Protein-Binding Agent

As mentioned herein, the present method uses a combination of small molecules and protein-binding agents to detect biomolecules, such as proteins, in a biological sample. In some embodiments, the method includes adding one or more protein-binding agents to the biological sample. In some embodiments, adding the protein-binging agent to the biological sample may generate a colloidal suspension.

In some embodiments, the one or more protein-binding agent may be added to the biological sample at a concentration of about 1 pg/ml to about 1 g/ml. Thus, in some embodiments, the one or more protein-binding agent may be added to the biological sample at a concentration of about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 200 μg/ml, about 300 μg/ml, about 400 μ/ml, about 500 μg/ml, about 600 μg/ml, about 700 μg/ml, about 800 μg/ml, about 900 μg/ml, about 1,000 μg/ml, about 2,000 μg/ml, about 3,000 μg/ml, about 4,000 μg/ml, about 5,000 μg/ml, about 6,000 μg/ml, about 7,000 μg/ml, about 8,000 μg/ml, about 9,000 μg/ml, about 10,000 μg/ml., about 11,000 μg/ml, about 12,000 μg/ml, about 13,000 μg/ml, about 14,000 μg/ml, about 15,000 μg/ml, about 16,000 μg/ml, about 17,000 μg/ml, about 18,000 μg/ml, about 19,000 μg/ml, about 20,000 μg/ml, about 10 μg/ml to about 10,000 μg/ml, about 20 μg/ml to about 10,000 μg/ml, about 30 μg/ml to about 10,000 μg/ml, about 40 μg/ml to about 10,000 μg/ml, about 50 μg/ml to about 10,000 μg/ml, about 60 μg/ml to about 10,000 μg/ml, about 70 μg/ml to about 10,000 μg/ml, about 80 μg/ml to about 10,000 μg/ml, about 90 μg/ml to about 10,000 μg/ml, about 100 μg/ml to about 10,000 μg/ml, about 200 μg/ml to about 9,000 μg/ml, about 300 μg/ml to about 8,000 μg/ml, about 400 μg/ml to about 7,000 μg/ml, about 500 μg/ml to about 6,000 μg/ml, about 600 μg/ml to about 5,000 μg/ml, about 700 μg/ml to about 4,000 μg/ml, about 800 μg/ml to about 3,000 μg/ml, about 900 μg/ml to about 2,000 μg/ml, about 950 μg/ml to about 1,500 μg/ml, about 10 μg/ml to about 1,000 μg/ml, about 20 μg/ml to about 1,000 μg/ml, about 30 μg/ml to about 1,000 μ/ml, about 40 μg/ml to about 1,000 μg/ml, about 50 μg/ml to about 1,000 μg/ml, about 60 μg/ml to about 1,000 μg/ml, about 70 μg/ml to about 1,000 μg/ml, about 80 μg/ml to about 1,000 μg/ml, about 90 μg/ml to about 1,000 μg/ml, about 10 μg/ml to about 900 μg/ml, about 10 μg/ml to about 800 μg/ml, about 10 μg/ml to about 700 μg/ml, about 10 μg/ml to about 600 μg/ml, about 10 μg/ml to about 500 μg/ml, about 10 μg/ml to about 400 μg/ml, about 10 μg/ml to about 300 μg/ml, about 10 μg/ml to about 200 μg/ml, about 10 μg/ml to about 100 μg/ml, about 10 μg/ml to about 50 μg/ml, about 1,000 μg/ml to about 20,000 μg/ml, about 2,000 μg/ml to about 19,000 μg/ml, about 3,000 μg/ml to about 18,000 μg/ml, about 4,000 μg/ml to about 17,000 μg/ml, about 5,000 μg/ml to about 16,000 μg/ml, about 6,000 μg/ml to about 15,000 μg/ml, about 7,000 μg/ml to about 14,000 μg/ml, about 8,000 μg/ml to about 13,000 μg/ml, about 9,000 μg/ml to about 12,000 μg/ml, about 9,000 μg/ml to about 11,000 μg/ml, or about 9,500 μg/ml to about 10,500 μg/ml. In some embodiments, about 1,000 μg/ml (0.1 mg/ml) of one or more protein-binding agent may be added to the biological sample. In some embodiments, about 2,000 μg/ml (0.2 mg/ml) of one or more protein-binding agent may be added to the biological sample.

The protein-binding agent may bind, interact with, or attract one or more proteins and/or their proteoforms in a biological sample. Thus, the protein-binding agent may also be referred to as a protein-interacting agent and/or a protein-attracting agent. A protein-binding agent may be any agent or material that provides a surface for protein binding. In other words, the protein-binding agent may have a surface capable of binding proteins and forming a protein corona on its surface.

The protein-binding agent may be an inorganic agent, metal-based agent, metal oxide-based agent, polymer-based agent, lipid-based agent, carbon-based agent, core-shell agent, composite agent, mesoporous agent, or a combination thereof. Additionally or alternatively, the protein-binding agent may include one or more nanoscale or microscale material.

A nanoscale material may be any material of which a single unit is sized (in at least one dimension) between about 1 nm and about 999 nm. For example, the nanoscale material (or nanomaterial) may be about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 999 nm. In a specific embodiment, the nanoscale material may be about an 80 nm nanoparticle.

A microscale material may be any material of which a single unit is sized (in at least one dimension) between about 1,000 nm and about 100,000 nm. For example, the microscale material (or micromaterial) may be about 1,000 nm, 2,000 nm, 3,000 nm, 4,000 nm, 5,000 nm, 6,000 nm, 7,000 nm, 8,000 nm, 9,000 nm, 10,000 nm, 20,000 nm, 30,000 nm, 40,000 nm, 50,000 nm, 60,000 nm, 70,000 nm, 80,000 nm, 90,000 nm, or 100,000 nm.

Thus, the protein-binding agent may be any material of which a single unit is sized (in at least one dimension) between about 1 nm and about 100,000 nm. For example, the protein-binding agent may be about 1 nm, about 50 nm, about 100 nm, about 500 nm, about 1,000 nm, about 5,000nm about 10,000 nm, about 50,000 nm, about 100,000 nm, about 50 nm to about 100,000 nm, about 100 nm to about 100,000 nm, about 500 nm to about 100,000 nm, about 1,000 nm to about 100,000 nm, about 5,000 nm to about 100,000 nm, about 10,000 to about 100,000 nm, about 50,000 to about 100,000 nm, about 1 nm to about 50,000 nm, about 1 nm to about 10,000 nm, about 1 nm to about 10,000 nm, about 1 nm to about 5,000 nm, about 1 nm to about 1,000 nm, about 1 nm to about 500 nm, about 1 nm to about 100 nm, about 1 nm to about 50 nm, about 50 nm to about 50,000 nm, about 100 nm to about 10,000 nm, or about 500 nm to about 5,000 nm.

A non-limiting list of nanoscale materials includes nanoparticles, nanorods, nanospheres, nanodisks, nanoclusters, nanofibers, and nanotubes. A non-limiting list of microscale materials includes microparticles, microrods, microspheres, and microbeads. The one or more protein-binding agent may include one or more nanoscale material, one or more microscale material, or a combination thereof. For example, in some embodiments, the one or more protein-binding agent may be a combination of one or more nanoparticles and one or more nanodisks. In certain embodiments, the one or more protein binding agent may be one or more nanoparticles.

The protein-binding agent may be made from organic materials, non-organic materials or combinations thereof. In some embodiments, the materials may be micelles, liposomes, iron oxide, graphene, silica, protein-based materials, polystyrene, silver, and gold materials, quantum dots, palladium, platinum, titanium, and combinations thereof.

In some embodiments, more than one protein-binding agent may include at least two to at least 1,000 protein-binding agents, which may be the same or different. In some embodiments, the number of protein-binding agents added to the biological sample may be any number to reach a desired concentration of protein-binding agents in the biological sample. In some embodiments, the number of protein-binding agents may be about 1, about 5, about 10, about 20, about 30, about 40, about 50, about 100, about 200, about 300, about 400, about 500, about 600, about 700, about 800, about 900, or about 1,000 per biological sample.

In some embodiments, the protein-binding agent used in the method may be all of the same type. For example, in some embodiments, the protein-binding agents added to the biological sample may be polystyrene nanoparticles. In other embodiments, the protein-binding agent may be different types. For example, the protein-binding agents added to the biological sample may be a mixture of polystyrene nanoparticles and silica microbeads. Any combination of protein-binding agents having a surface to bind proteins may be used herein. The protein-binding agent selection is not of particular importance as long as it is able to form a protein corona on its surface. One skilled in the art can determine one or more suitable materials capable of forming a protein corona on its surface.

In some embodiments, the protein-binding agent may have a polydispersity index (PDI) of about 0.01 to about 10. Thus, the protein-binding agent may have a PDI of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.02 to about 0.6, about 0.03 to about 0.5, about 0.04 to about 0.4, 0.05 to about 0.3, 0.06 to about 0.2, 0.07 to about 0.1, 0.08 to about 0.09, about 0.01 to about 0.6, about 0.01 to about 0.5, about 0.01 to about 0.4, about 0.01 to about 0.3, about 0.01 to about 0.2, about 0.01 to about 0.1, about 0.01 to about 0.09, about 0.01 to about 0.08, about 0.01 to about 0.07, about 0.01 to about 0.06, about 0.01 to about 0.05, about 0.01 to about 0.04, about 0.01 to about 0.03, about 0.01 to about 0.02, about 0.02 to about 0.7, about 0.03 to about 0.7, about 0.04 to about 0.7, about 0.05 to about 0.7, about 0.06 to about 0.7, about 0.07 to about 0.7, about 0.08 to about 0.7, about 0.09 to about 0.7, about 0.1 to about 0.7, about 0.2 to about 0.7, about 0.3 to about 0.7, about 0.4 to about 0.7, about 0.5 to about 0.7, or about 0.6 to about 0.7. A PDI of about 0.01 may be referred to as mono-dispersed.

In some embodiments, the PDI of the protein-binding agent may be about 0.01 to about 0.7. In a specific embodiment, the protein-binding agent PDI may be about 0.7. In another embodiment, the protein-binding agent PDI may be about 0.3. In another embodiment, the protein-binding agent PDI may be about 0.2.

The one or more protein-binding agent has a surface capable of binding proteins to the biological sample and allows a protein corona to form on the surface of the one or more protein-binding agent. This may produce a complex including the protein corona and the one or more protein-binding agent. Thus, the protein-binding agent may also be referred to herein as a protein-corona forming agent or a protein-corona attracting agent.

### Protein Corona

The disclosed method includes adding a protein-binding agent to a biological sample to generate a protein corona. In some embodiments, the protein corona may form on a protein-binding agent spontaneously upon addition of one or more protein-binding agents to a biological sample. In some embodiments, the protein corona may form on a protein-binding agent upon addition of one or more protein-binding agent to a biological sample, followed by incubation of said biological sample containing one or more protein-binding agent. Thus, in some embodiments, the protein-binding agent and biological sample may be incubated for a time which allows a protein corona to form on the surface of the one or more protein-binding agent. In particular embodiments, a protein corona may be formed around a nanoparticle.

Although protein coronas may include mostly proteins, in some embodiments, the protein corona may include molecules in addition to proteins, such as lipids and other biological sample components, that are able to bind to a protein-binding agent as described herein. In this case, the protein corona may be referred to as a biomolecule corona. In some embodiments, the proteins in the protein corona may be present in the same ratio as the proteins in the untreated biological sample (i.e., the biological sample before addition of small molecules and/or protein-binding agents). In some embodiments, the proteins in the protein corona may be present in a different ratio than the proteins in the untreated biological sample. In some embodiments, the protein corona profile may be different in different subjects and/or in different biological samples. In some embodiments, the protein corona profile may be the same in different subjects and/or in different biological samples.

In some embodiments, a protein corona may form in different patterns and have different compositions depending on the protein and/or protein-binding agent size, shape, composition, charge, and surface functional groups. In some embodiments, protein corona properties may vary in different environmental factors such as temperature, pH, shearing stress, immersed media composition, and exposing time. In some embodiments, protein corona compositions may change according to the biochemical and physiochemical surface interactions with the protein-binding agent. In some embodiments, the protein corona may include a hard corona and/or a soft corona. The hard corona may include higher-affinity proteins that may be irreversibly bonded to the protein-binding agent's surface and/or to other proteins in the protein corona. The soft corona may include lower-affinity proteins that are reversibly bound to the protein-binding agent's surface and/or to other proteins in the protein corona. In some embodiments, proteins in the soft corona may be exchanged or detached over time. In some embodiments, larger proteins with lower affinities may aggregate to the protein-binding agent's surface first, and over time, smaller proteins with higher affinities may replace them (i.e., “hardening” the corona).

Additionally or alternatively, protein corona compositions may change upon adding the one or more small molecules to a biological sample along with one or more protein-binding agent as described herein. For example, the addition of phosphatidylcholine to a biological sample along with one or more protein-binding agents may alter protein corona composition. For example, phosphatidylcholine may itself bind specific proteins based off its properties, thus changing the type and/or number of proteins that may bind to the protein-binding agents. For example, a small molecule may reduce the amount of one or more proteins that are free-floating in the biological sample. Thus, in some embodiments, the type and/or number of proteins available to bind a protein-binding agent may be altered by one or more small molecules in the biological sample. In some embodiments, phosphatidylcholine may bind high-abundance proteins, such as albumin, thus altering the protein corona composition to include less high-abundance proteins, such as albumin.

In some embodiments, protein coronas may be used to detect one or more protein types and/or the amount of one or more protein types present in a biological sample.

### Method for Preparing Complex for Detecting Proteins

Also described herein is a method of preparing the complex including the protein corona and the protein-binding agent for detecting the proteins and/or their proteoforms in the protein corona. In some embodiments, the complex may be separated from the remainder of the biological sample, for example, by centrifugation (such as gradient centrifugation), size exclusion chromatography, magnetic separation, field-flow fractionation, etc. In some embodiments, the complex may be washed and resuspended. In some embodiments, the proteins from the complex may be reduced, alkylated, and/or digested. In some embodiments, the proteins and/or proteoforms that were present in the protein corona may be detected by an antibody-based technique. For example, the proteins and/or their proteoforms may be detected using Western blotting, enzyme-linked immunosorbent assay (ELISA), or OLINK® (olink.com). Additionally or alternatively, the proteins and/or their proteoforms may be detected by a proteomics technique. For example, the proteins and or their proteoforms may be detected by top-down, middle-down, or bottom-up proteomics, such as LC-MS/MS, or a combination thereof. In some embodiments, top-down, middle-down, or bottom-up proteomics may be based on Data-Dependent Acquisition (DDA) or Data-Independent Acquisition (DIA). In some embodiments, DDA or DIA may include label-free or labeling-based techniques such as tandem mass tag (TMT) labeling, dimethyl labeling, etc. In some embodiments, one or more protein-binding agents may be used in a protein corona sensor array. In some embodiments, multiple biological samples may be tested and compared.

In some embodiments, proteins and/or their proteoforms in the hard protein corona, soft protein corona, or a combination of the hard and soft protein corona may be detected. In some embodiments, proteins present in the protein corona may be detected after a biological sample has been incubated with one or more small molecule and one or more protein-binding agent. In some embodiments, the protein corona composition may change over time. For example, in some embodiments, a protein corona composition after 10 minutes of incubation may be different than a protein corona composition after 20 minutes of incubation. In some embodiments, molecules other than proteins may be detected that have been bound to the protein-binding agent.

### Increased Protein Detection

In some embodiments, the method described herein may detect more proteins and/or their proteoforms compared to using a biological sample without the one or more protein-binding agent and/or the one or more small molecule. In other words, the present method may result in an increase in the number of detected proteins compared to using a biological sample without the one or more protein-binding agent and/or the one or more small molecule or small molecule combination. In some embodiments, the method may detect more proteins and/or their proteoforms compared to using a biological sample including the one or more protein-binding agent but not including the one or more small molecule or small molecule combination. For example, in some embodiments, when using the disclosed method, there may be at least a 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 0.1 to 20, 0.2 to 20, 0.3 to 20, 0.4 to 20, 0.5 to 20, 0.6 to 20, 0.7 to 20, 0.8 to 20, 0.9 to 20, 1 to 20, 2 to 19, 3 to 18, 4 to 17, 5 to 16, 6 to 15, 7 to 14, 8 to 13, 9 to 12, 10 to 11, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20-fold increase in the number of proteins and/or their proteoforms compared to a biological sample without the one or more protein-binding agent and the one or more small molecule or small molecule combination. In some embodiments, when using the disclosed method, there may be at least a 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 0.1 to 20, 0.2 to 20, 0.3 to 20, 0.4 to 20, 0.5 to 20, 0.6 to 20, 0.7 to 20, 0.8 to 20, 0.9 to 20, 1 to 20, 2 to 19, 3 to 18, 4 to 17, 5 to 16, 6 to 15, 7 to 14, 8 to 13, 9 to 12, 10 to 11, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20-fold increase in the number of proteins and/or their proteoforms compared to a biological sample including the one or more protein-binding agent but not including the one or more small molecule or small molecule combination. In a specific embodiment, there may be at least an 8-fold increase in the number of proteins and/or their proteoforms compared to a biological sample without the one or more protein-binding agent and the one or more small molecule. In a specific embodiment, there may be at least a 3-fold increase in the number of proteins and/or their proteoforms compared to a biological sample without the one or more protein-binding agent and the one or more small molecule. In some embodiments, there may be at least a 2-fold increase in the number of proteins and/or their proteoforms compared to a biological sample comprising the one or more protein-binding agent without the addition of the one or more small molecule.

In some embodiments, the disclosed method may increase the detection of low-abundance proteins. For example, one or more of the small molecules added to the biological sample may bind one or more high-abundance proteins, such as albumin. Thus, in some embodiments, less high-abundance proteins may be present in the biological sample to bind the protein-binding agents. So in some embodiments, more low-abundance proteins may attach to the protein-binding molecule and may be detected by the disclosed method. Thus, in some embodiments, when detecting proteins in the protein corona, adding one or more small molecules to the biological sample with one or more protein-binding agents may reduce the detection of one or more high-abundance protein by at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In some embodiments, to detect more than one protein and/or their proteoforms, multiple protein-binding agent types may be used. In some embodiments, different protein-binding agents may attract different protein types. In some embodiments, using more than one type of protein-binding agent may increase the number of detected proteins in a biological sample. In some embodiments, when more than one protein-binding agent is used, each protein-binding agent type may have distinct physicochemical properties. Thus, in some embodiments, the protein corona formed around the protein-binding agents may be different for different protein-binding agents.

Thus, the present disclosure provides a method of increasing the number of proteins detected in a biological sample compared to other known methods.

Also described herein is a composition for use in detecting proteins and/or their proteoforms. The composition may include one or more small molecules as described herein, one or more protein-binding agents as described herein, and one or more biological sample as described herein.

Along with detecting proteins in a biological sample, the present disclosure also provides a method for detecting biomarkers in a biological sample.

### Method for Detecting Biomarkers

In another embodiment, the present disclosure provides a method for detecting one or more biomarkers or a pattern of one or more biomarkers in a biological sample. In some embodiments, the one or more biomarkers or pattern of one or more biomarkers may be associated with a health spectrum condition, such as a disease or disorder. In some embodiments, the method may include adding one or more small molecules, as described herein, to at least two biological samples, where each biological sample may be from different subjects diagnosed with the disease. The method may also include adding one or more protein-binding agent (which may all be the same type or a combination of different types), as described herein, having a surface capable of binding proteins to the biological samples. In some embodiments, a portion corona is allowed to form on the surface of the protein-binding agent to produce a complex comprising the protein corona and the one or more protein-binding agents.

The method may also include detecting the one or more biomarker or the pattern of one or more biomarkers in the protein corona by an antibody-based technique or a proteomics technique, as described herein. In some embodiments, a biomarker pattern may include multiple types of biomarkers. In some embodiments, a biomarker pattern may include a certain amount of one or more biomarkers in a biological sample. In some embodiments, individual organisms or biological samples may have different biomarker patterns. For example, a biomarker pattern, similar to a biomarker, may be used to detect and/or diagnose a disease or disorder in a subject.

A biomarker may be a measurable indicator of some biological state or condition. In some embodiments, biomarkers may be upregulated or downregulated according to different disease types or disease stages. In some embodiments, the biomarker may be a molecular, physiologic, histologic, and/or radiographic biomarker. Additionally or alternatively, the biomarker may be predictive, prognostic, or diagnostic. In some embodiments, predictive biomarkers may help optimize ideal treatments. Examples of predictive biomarkers may include HER2/neu in breast cancer or EGFR1 mutations in non-small cell lung cancer. In some embodiments, diagnostic biomarkers can be a traceable substance that is introduced into an organism as a means to examine aspects of health. In other embodiments, a diagnostic biomarker may be used as a substance whose detection indicates a particular disease state. For example, the presence of an antibody may indicate an infection. For example, a diagnostic biomarker may be prostate-specific antigen (PSA), which may be used as a proxy of prostate size with rapid changes potentially indicating cancer.

In some embodiments, to detect more than one biomarker and/or more than one biomarker pattern, multiple protein-binding agent types may be used. In some embodiments, different protein-binding agents may attract different biomarker types. In some embodiments, using more than one type of protein-binding agent may increase the number of detected biomarkers in a biological sample.

In some embodiments, the health spectrum condition may be any health state, including complete well-being, minor health issues, chronic conditions, and severe illnesses. The health spectrum refers to overall well-being and the factors that influence it, whether they lead to optimal health or contribute to illness. In some embodiments, a health spectrum may include a condition a subject may have which is not considered a disease or disorder yet for medical treatment. For example, in some embodiments, a health spectrum condition may include a predisposition for a disease or disorder. In other words, the presence of biomarker(s) may indicate a subject is at risk of developing a health spectrum condition and/or a disease/disorder. In some embodiments, the biomarker(s) for a disease/disorder may be the same for a disease or disorder predisposition. Additionally or alternatively, the biomarker(s) for a disease/disorder may be different for the same disease or disorder. Alternatively, the health spectrum condition may be a disease or a disorder. Some health spectrum condition examples include, but are not limited to, a predisposition to, or risk of developing, or diagnosed obesity, heart disease, liver disease, kidney disease, depression, cancer, etc.

In some embodiments, the disease may be any condition that adversely affects the structure or function of all or part of an organism and is not immediately due to any external injury. In some embodiments, the disease the biomarker may be associated with may be a neoplastic disease, cardiovascular disease, metabolic disease, infectious disease, inflammatory disease, congenital and hereditary diseases, degenerative disease, a neurological disease, and a combination thereof. For example, in some embodiments, the disease may be a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof. In some embodiments, the disease may be a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

Suitable biomarkers for Alzheimer's disease include, but are not limited to, for example, the amyloid beta (Aβ)42/40 ratio, phosphorylated tau (p-tau), serum neurofilament light chain (NfL), and glial fibrillary acidic protein (GFAP).

Suitable cancer biomarkers include, but are not limited to, for example, AHSG (α2-HS-Glycoprotein), AKR7A2 (Aflatoxin B1 aldehyde reductase), AKT3 (PKB γ), ASGR1 (ASGPR1), BDNF, BMP1 (BMP-1), BMPER, C9, CA6 (Carbonic anhydrase VI), CAPG (CapG), Carcino-embryonic antigen, CDH1 (Cadherin-1), CHRDL1 (Chordin-Like 1), CKB-CKM-(CK-MB), CLIC1 (chloride intracellular channel 1), CMA1 (Chymase), CNTN1 (Contactin-1), COL18A1 (Endostatin), CRP, CTSL2 (Cathepsin V), DDC (dopa decarboxylase), EGFR (ERBB1), FGA-FGB-FGG (D-dimer), FN1 (Fibronectin FN1.4), GHR (Growth hormone receptor), GPI (glucose phosphate isomerase), HMGB1 (HMG-1), HNRNPAB (hnRNP A/B), HP (Haptoglobin, Mixed Type), HSP90AA1 (HSP 90α), HSPA1A (HSP 70), IGFBP2 (IGFBP-2), IGFBP4 (IGFBP-4), IL12B-IL23A (IL-23), ITIH4 (Inter-α-trypsin inhibitor heavy chain H4), KIT (SCF sR), KLK3 -SERPINA3 (PSA-ACT), LICAM (NCAM-L1), LRIG3, MMP12 (MMP-12), MMP7 (MMP-7), NME2 (NDP kinase B), PA2G4 (ErbB3 binding protein Ebp1), PLA2G7 (LpPLA2/PAFAH), PLAUR (suPAR), PRKACA (PRKA C-α), PRKCB (PKC-ß-II), PROK1 (EG-VEGF), PRSS2 (Trypsin-2), PTN (Pleiotrophin), SERPINA1 (α1-Antitrypsin), STC1 (Stanniocalcin-1), STX1A (Syntaxin 1A), TACSTD2 (GA733-1 protein), TFF3 (Trefoil factor 3), TGFBI (βIGH3), TPI1 (Triosephosphate isomerase), TPT1 (Fortilin), YWHAG (14-3-3 protein γ), YWHAH (14-3-3 protein eta), prostate cancer biomarkers, for example, p63 protein, PSA, Pro-PSA, Pro2PSA, PHI, PCA3, TMPRSS3:ERG, PCMT, MTEN, breast cancer markers, for example, epidermal growth factor receptor 2 (HER2) oncogene, melanoma biomarker BRAF, lung cancer biomarker EML4-ALK, A2ML1, BAX, C10orf47, Clorfl62, CSDA, EIFC3, ETFB, GABARAPL2, GUKI, GZMH, HIST1H3B, HLA-A, HSP90AA1, NRGN, PRDX5, PTMA, RABACI, RABAGAP1L, RPL22, SAP 18, SEPW1, SOX1, EGFR, EGFRvIII, apolipoprotein A1, apolipoprotein CIII, myoglobin, tenascin C, MSH6, claudin-3, claudin-4, caveolin-1, coagulation factor III, CD9, CD36, CD37, CD53, CD63, CD81, CD136, CD147, Hsp70, Hsp90, Rabl3, Desmocollin-1, EMP-2, CK7, CK20, GCDF15, CD82, Rab-5b, Annexin V, MFG-E8, HLA-DR, a miR200 microRNA, MDC, NME-2, KGF, PIGF, F1t-3L, HGF, MCP1, SAT-1, MIP-1-b, GCLM, OPG, TNF RII, VEGF-D, ITAC, MMP-10, GPI, PPP2R4, AKR1B1, Amy1A, MIP-1b, P-Cadherin, EPO and the like. For example, biomarkers for breast cancer include, but are not limited to, Circulating Tumor Cells (EpCAM, CD45, cytokeratins 8, 18+, 19+), ER/PR, HER-2/neu, CA15-3, CA27.29, and the like. Biomarkers for colorectal cancer include, but are not limited to, for example, EGFR, KRAS, UGT1A1, Fibrin/fibrinogen degradation product (DR-70), Human hemoglobin (fecal occult blood), and the like. Biomarkers associated with leukemia/lymphoma include, but are not limited to, e.g., CD20 antigen, CD30, FIP1L1-PDGFRalpha, PDGFR, Philadelphia Chromosome (BCR/ABL), PML/RAR alpha, TPMT, UGT1A1, and the like. Biomarkers associated with lung cancer include but are not limited to, e.g., ALK, EGFR, KRAS and the like. Biomarkers associated with ovarian cancer include but are not limited to, e.g., ROMA (HE4+CA-125), OVA1 (multiple proteins), HE4, CA-125, and the like. Biomarkers associated with hepatocellular cancer include but are not limited to AFP-L3%, and the like. Biomarkers associated with gastrointestinal stromal tumors include but are not limited to c-Kit, and the like. Biomarkers associated with pancreatic cancer include but are not limited to CA19-9, and the like. Biomarkers are known in the art, and can be found in, for example, Bigbee W, Herberman R B. Tumor markers and immunodiagnosis. In: Bast R C Jr., Kufe D W, Pollock R E, et al., editors. Cancer Medicine. 6th ed. Hamilton, Ontario, Canada: BC Decker Inc., 2003; Andriole G, Crawford E, Grubb R. et al. Mortality results from a randomized prostate-cancer screening trial. New England Journal of Medicine 2009; 360(13):1310-1319; Schroder F H, Hugosson J, Roobol M J, et al. Screening and prostate-cancer mortality in a randomized European study. New England Journal of Medicine 2009; 360(13):1320-1328; Buys S S, Partridge E, Black A. et al. Effect of screening on ovarian cancer mortality: the Prostate, Lung, Colorectal and Ovarian (PLCO) Cancer Screening Randomized Controlled Trial. JAMA 2011; 305(22):2295-2303; Cramer D W, Bast R C Jr, Berg C D, et al. Ovarian cancer biomarker performance in prostate, lung, colorectal, and ovarian cancer screening trial specimens. Cancer Prevention Research 2011; 4(3):365-374; Sparano J A, Gray R J, Makower D F, et al. Prospective validation of a 21-gene expression assay in breast cancer. New England Journal of Medicine 2015; First published online Sep. 28, 2015. doi: 10.1056/NEJMoa1510764, and clinicalproteomicsjournal.biomedcentral.com/articles/10.1186/1559-0275-10-13/tables/1 incorporated by reference in their entireties.

Biomarkers associated with a cardiovascular disease may include, but are not limited to, lipid profile, glucose, and hormone level and physiological biomarkers based on measurement of levels of important biomolecules such as serum ferritin, triglyceride to HDLp (high density lipoproteins) ratio, lipophorin-cholesterol ratio, lipid-lipophorin ratio, LDL cholesterol level, HDLp and apolipoprotein levels, lipophorins and LTPs ratio, sphingolipids, Omega-3 Index, and ST2 level, among others. Suitable biomarkers for cardiovascular disease can be found in the art, for example, but not limited to, in van Holten et al. “Ciculating Biomarkers for Predicting Cardiovascular Disease Risk; a Systemic Review and Comprehensive Overview of Meta-Analyses” PLOS One, 2013 8(4): e62080, incorporated by reference in its entirety.

Biomarkers associated with a neurological disease may include, but are not limited to, e.g., Aβ1-42, t-tau and p-tau 181, α-synuclein, among others. See, e.g., Chintamaneni and Bhaskar “Biomarkers in Alzheimer's Disease: A Review” ISRN Pharmacol. 2012. 2012:984786. Published online 2012 Jun. 28, incorporated by reference in its entirety.

Also described herein is a composition for use in detecting one or more biomarkers or a pattern of one or more biomarkers, where the composition may include one or more small molecule, one or more protein-binding agent, and one or more biological sample.

### Method for Diagnosing a Disease or Identifying a Health Spectrum Condition

In another embodiment, a method of diagnosing a disease or identifying another health spectrum condition, such as a predisposition for a disease in a subject is disclosed herein. The method may include adding one or more small molecule, as described herein, to a biological sample, as described herein, from the subject, and adding one or more protein-binding agent, as described herein, to the biological sample. The protein-binding agent has a surface capable of binding proteins, and a protein corona is allowed to form on the surface of the one or more protein-binding agent. Thus, a complex comprising the protein corona and the one or more protein binding agent may be formed. The method includes detecting one or more biomarker or a pattern of one or more biomarker, as described herein, associated with the disease in the protein corona by an antibody-based technique, as described herein, and/or a proteomics technique, as described herein.

As described above, the “health spectrum” covers a range of health states, including complete well-being, minor health issues, chronic conditions, and severe illnesses. For example, some health spectrum condition examples include both mental and physical conditions at a pre-disease or pre-disorder level all the way to severe illness, such as, but are not limited to, obesity, heart disease, liver disease, kidney disease, depression, cancer, etc.

As described above, the disease may be a neoplastic disease, cardiovascular disease, metabolic disease, infectious disease, inflammatory disease, congenital and hereditary diseases, degenerative disease, a neurological disease, and a combination thereof. For example, in some embodiments, the disease may be a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof. In some embodiments, the disease may be a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

In some embodiments, the disclosed method may be used to provide a predisposition (e.g. a chance or likelihood) of developing the disease. In some embodiments, the disclosed method may be used to detect early onset of a disease (i.e., early disease diagnosis). For example, in some embodiments, apolipoproteins may be detected, which are indicators of cardiovascular and neurodegenerative disorders. Thus, the present method may detect protein categories that are important in disease onset and progression.

As mentioned herein, biomarkers(s) for a disease/disorder may be the same and/or different for a predisposition of a disease/disorder. Thus, in some embodiments, the same biomarkers that are detected for diagnosing a disease/disorder may be detected to identify a predisposition for that disease/disorder. In some embodiments, the biomarker concentration or amount may be lower in the sample for identifying a predisposition for a disease compared to diagnosing the disease. In some embodiments, diagnosing a disease, or identifying another health spectrum condition, such as a predisposition for a disease or health spectrum condition may help prevent or reduce the risk of developing conditions such as obesity, heart disease, liver disease, kidney disease, depression, cancer, etc. Thus, in some embodiments, subjects diagnosed with a disease, a health spectrum condition, or identified as having a predisposition for a disease or health spectrum condition may take measures to prevent or slow the progression of the disease or condition.

Also described herein is a composition for use in diagnosing a disease, where the composition may include one or more small molecule, one or more protein-binding agent, and one or more biological samples (combined in any order).

### EXAMPLES

The following examples are merely illustrative, and do not limit this disclosure in any way.

### Example 1: Addition of Various Small Molecules and Small Molecule Combinations Alters Nanoparticle Protein Corona

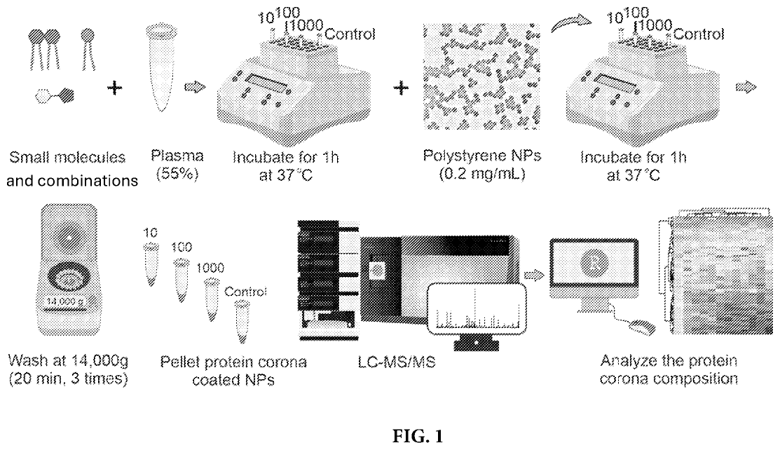

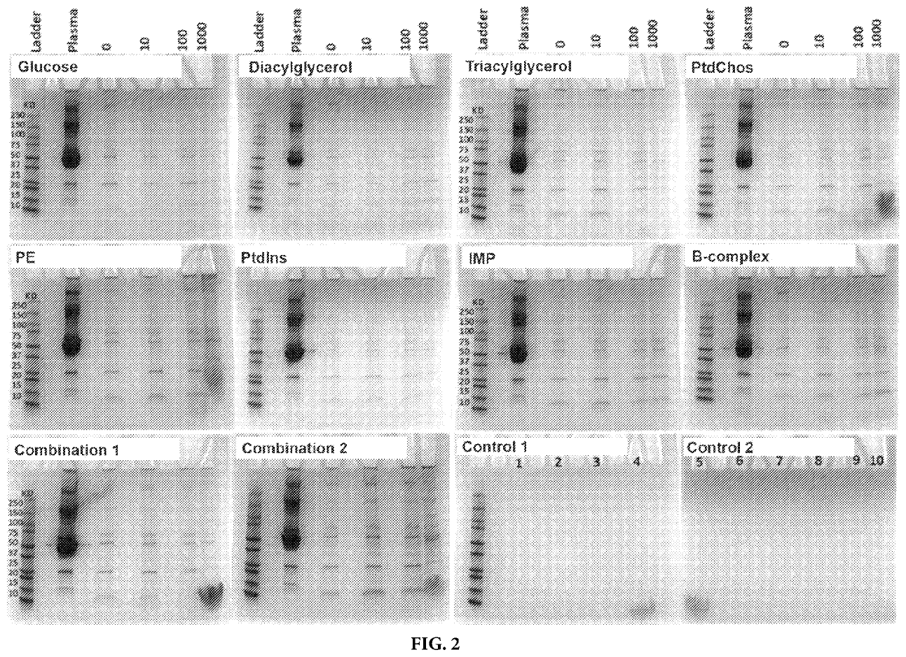

An overview of the process is outlined in FIG. 1. Generally, exposing small molecules to human plasma, nanoparticles (NPs) were incubated with human plasma, purified and isolated, and used for analysis of the protein corona profile on the surface of the NPs using SDS-PAGE and liquid chromatography-mass spectrometry (LC-MS/MS).

### Materials

Healthy human plasma protein was obtained from Innovative Research®, Inc. (Novi, MI) (and diluted to a final concentration of 55% using phosphate buffer saline (PBS, 1X).

Commercially available plain polystyrene nanoparticles (NPs), averaging about 80 nm in size, were provided by Polysciences®, Inc. (Warrington, PA).

Small molecules were ordered from Millipore Sigma [phosphatidylcholine (PtdChos), phosphatidylethanolamine (PE), L-α-phosphatidylinositol (PtdIns), inosine 5′-monophosphate (IMP)], Abcam® [triacylglycerols], Fisher Scientific® [diacylglycerols and glucose], VWR® [vitamin B complex], and diluted with PBS (1X) to the desired concentration. Eight distinct sets of small molecules, namely glucose, triacylglycerols, diacylglycerols, phosphatidylcholine (PtdChos), phosphatidylethanolamine (PE), L-α-phosphatidylinositol (PtdIns), inosine 5′-monophosphate (IMP), and vitamin B complex, were assessed to determine their effect on the protein corona formed around polystyrene NPs. The selection of these small molecules was based on their ability to interact with a broad spectrum of proteins, which significantly influences the composition of the protein corona surrounding NPs. For example, vitamin B complex components can interact with a wide range of proteins including albumin, hemoglobin, myoglobin, pantothenate permease, acyl carrier protein, lactoferrin, prion, β-amyloid precursor, and niacin-responsive repressor. Additionally, to assess the collective effects of these molecules, two combinations of small molecules were analyzed. Small molecule combination 1 is a blend of glucose, triacylglycerols, diacylglycerols, and PtdChos, and small molecule combination 2 is a blend of PE, PtdIns, IMP, and vitamin B complex.

### Protein Corona Formation on the Surface of NPs in the Presence of Small Molecules

For protein corona formation in the presence of small molecules, commercial pooled healthy human plasma diluted to 55% by PBS was first incubated with the individual small molecules or a combination of small molecules at varying concentrations (i.e., 10, 100, and 1,000 μg/ml) for 1 hour at 37° C., allowing the small molecules to interact with the biological matrix. The small molecule combinations included each of the four small molecules (glucose, triacylglycerols, diacylglycerols, and PtdChos for combination 1; PE, PtdIns, IMP, and vitamin B complex for combination 2) at the given concentration. For example, combination 1 at 10 μg/ml included glucose at 10 μg/ml, triacylglycerols at 10 μg/ml, diacylglycerols at 10 μg/ml, and PtdChos at 10 μg/ml). Additionally, for example, combination 2 at 1,000 μg/ml included 1,000 μg/ml PE, 1,000 μg/ml PtdIns, 1,000 μg/ml IMP, and 1,000 μg/ml vitamin B complex. Subsequently, polystyrene NPs were added to the solution containing plasma and small molecule(s) so that the final concentration of the NPs was 0.2 mg/ml, and the solution was incubated for another 1 hour at 37° C. with agitation. These methods allow the formation of a distinct protein corona around the NPs. All experimental trials were designed in a way that the concentration of the NPs, human plasma, and small molecules were 0.2 mg/ml, 55%, and 10, 100, and 1,000 μg/ml, respectively. To remove unbound proteins in the plasma or proteins only loosely attached to the surface of the NPs, protein-NPs complexes were centrifuged at 14,000xg for 20 minutes, NP pellets were washed three times with cold PBS and centrifuged under the same conditions, and the final pellet was collected for further analysis. For the PtdChos concentration study, various concentrations for PtdChos (i.e., 100, 500, 1,000, and 10,000 μg/ml) were used in the above protocol for preparation of the samples for mass spectrometry analysis.

### NP Characterization

Dynamic light scattering (DLS) and zeta potential analysis were performed to measure the size distribution and surface charge of the NPs before and after protein corona formation using a Zetasizer® ZS90 nano series DLS instrument (Malvern Panalytical®). A helium-neon (He—Ne) laser with a wavelength of 632 nm was used for size distribution measurement at room temperature. Transmission electron microscopy (TEM) was carried out using a JEM-2200FS field emission electron microscope (JEOL® Ldt.) operated at 200 kV. The instrument was equipped with an in-column energy filter and an Oxford Instruments Energy Dispersive X-ray spectrometry (EDXS) system. 20 μl of the bare NPs were deposited onto a copper grid and used for imaging. For protein corona-coated NPs, 20 μl of sample was negatively stained using 20 μl uranyl acetate 1%, washed with deionized (DI) water, deposited into a copper grid, and used for imaging.

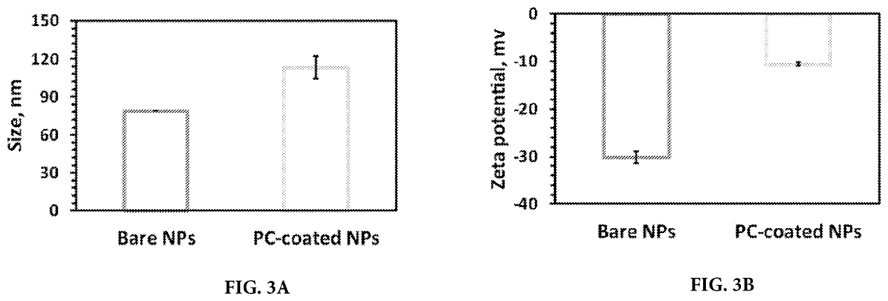

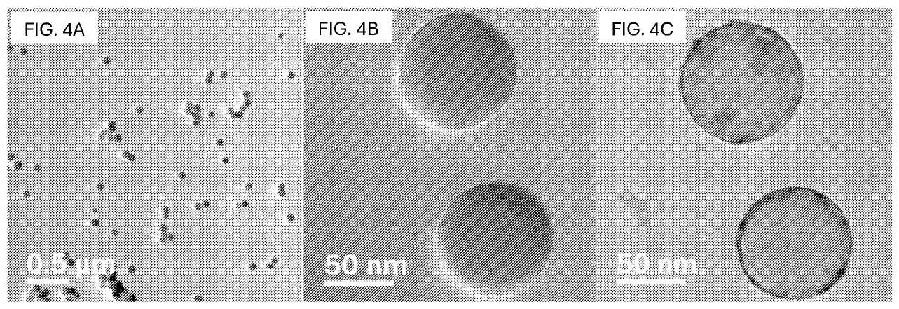

FIG. 3A and FIG. 3B show dynamic light scattering (DLS), zeta potential, and transmission electron microscopy (TEM) analyses for both the untreated NPs and those covered by a protein corona. The untreated polystyrene NPs exhibited excellent monodispersity, with an average size of 78.8 nm with the polydispersity index of 0.026 and a surface charge of —30.1±0.6 mV (FIG. 3A and FIG. 3B). Upon formation of the protein corona, the average size of NPs expanded to 113 nm, and the surface charge shifted to −10 mV±0.4 mV (FIG. 3A and FIG. 3B). TEM analysis further corroborated the size and morphology alterations of the NPs before and after protein corona formation. The Polydispersity Index (PDI) of bare protein corona-coated NPs was found to be 0.023 and 0.214, respectively (FIG. 4A-4C).

### Initial Protein Corona Profiles

The initial protein corona profiles of the NPs in the presence of small molecules and small molecule combinations were studied using SDS-PAGE analysis (FIG. 2). For SDS-PAGE analysis, 20 μl of protein corona coated NPs were combined with 20 μl of 2x Laemmli sample buffer, heated at 85° C. for 7 minutes, and loaded into precast gels. Following gel electrophoresis, the gels were fixed in a solution containing 10% acetic acid and 40% ethanol, then stained overnight with 50 mL of Coomassie blue stain. Following staining, the gels were washed and scanned. The results demonstrate that at certain concentrations for particular small molecules, the protein band intensities are different. For example, the protein corona profile significantly changes in the presence of vitamin B complex molecules (FIG. 2). More specifically, at high levels of vitamin B complex molecules, the protein corona profile reveals new protein band intensities (about 50 kDa) which were absent in normal protein corona of the NPs or lower concentration of vitamin B complex molecules. In the presence of high levels of triacylglycerol, some protein bands disappeared or had reduced intensity, indicating the crucial effect of small molecules and their concentrations on the protein corona profile of NPs. To investigate how spiking different concentrations of small molecules can influence the molecular composition of the protein corona, protein corona composition was also determined using LC-MS/MS.

### LC-MS/MS Sample Preparation for the Screening and Concentration Series Experiments

Initial processing: NPs coated with protein corona were first washed with PBS and then resuspended in 30 μl of PBS, enriched with 15 mM phosphate (pH 7.4). The bound total protein content was estimated to be around 1 μg per sample, determined via a Micro BCA™ assay (Thermo Scientific™). The samples were reduced with 2 mM dithiothreitol (DTT) incubated at 50° C. for 45 minutes with shaking at 700 rpm. Thereafter, proteins were alkylated with iodoacetamide (IAA) at 8 mM in the dark at room temperature. LysC/Trypsin enzyme mix was added at a concentration of 0.02 μg/μl and samples were incubated overnight at 37° C.

Samples were centrifuged at 16,000xg for 20 minutes at room temperature to pellet the NPs. The supernatant, containing the peptide digest, was collected, vacuum dried, and desalted using Pierce™ C18 Spin Tips (Thermo Scientific™, catalog number: 84850) following the manufacturer's instructions. Samples were vacuum dried again and stored at −80° C. until LC-MS/MS analysis.

LC-MS/MS Analysis: Dried samples were reconstituted with 1 μg of peptides in 25 μl of liquid chromatography (LC) loading buffer (3% acetonitrile (ACN), 0.1% trifluoroacetic acid (TFA)) and analyzed using LC-MS/MS. A 60-minute gradient was applied in the label-free quantification (LFQ) mode, with 5 μl aliquots injected in triplicate. Control samples (55% human plasma) were prepared with 8 μg of peptides in 200 μl of loading buffer and analyzed similarly. An UltiMate™ 3000 RSLCnano (Thermo Scientific™) high-performance liquid chromatography system was used with predefined columns, solvents, and gradient settings. Data Dependent Analysis (DDA) was performed with specific MS (also known as MS¹) and MS/MS (also known as MS²) scan settings, followed by data analysis using Proteome Discoverer™ 2.4 (Thermo Scientific™), applying the protocols detailed in Ashkarran et al., 2022. The PdtChos concentration series experiment was performed using the same protocol, and the samples were analyzed over a 120 minute gradient.

### Protein Corona and Small Molecules Enable Deep Profiling of the Plasma Proteome

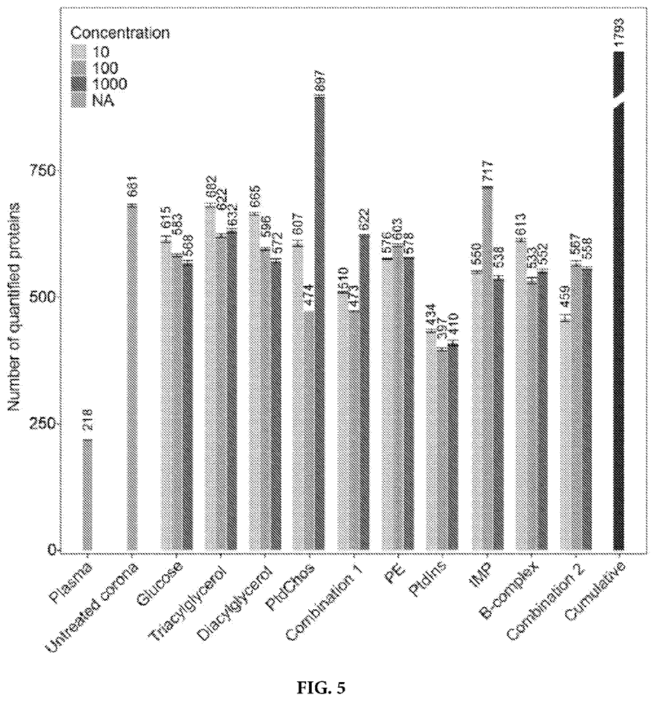

To investigate how spiking different concentrations of small molecules can influence the molecular composition of the protein corona, samples were subjected to high-resolution LC-MS/MS analysis. While the analysis of plasma alone led to quantification of 218 unique proteins, analysis of the protein corona formed on the polystyrene NPs significantly enhanced the depth of plasma proteome sampling to enable quantification of 681 unique proteins. Furthermore, the inclusion of small molecules further deepened plasma proteome sampling to enable quantification of between 397 and up to 897 unique proteins, depending on the small molecules added to plasma prior to corona formation. When comparing the use of protein coronas, both with and without the inclusion of small molecules, to the analysis of plasma alone (FIG. 5), there is a notable increase—approximately a 3-fold rise—in the number of proteins that can be quantified.

Interestingly, the concentration of small molecules did not significantly affect the number of quantified proteins; only a small stepwise reduction in the number of quantified proteins was noted with increasing concentrations of glucose and diacylglycerol. Cumulatively, the incorporation of small molecules and small molecule combinations into the protein corona of NPs led to a significant increase in protein quantification, with a total of 1793 proteins quantified, marking an 8.25-fold increase compared to plasma alone. Specifically, the addition of small molecules resulted in the quantification of 1573 additional proteins compared to plasma alone, and 1037 more proteins than the untreated protein corona. Strikingly, spiking 1000 μg/ml of PtdChos singlehandedly increased the number of quantified proteins to 897 (4.1 fold of quantified proteins in plasma alone, and 1.3-fold of quantified proteins in untreated corona). This observation prompted a detailed investigation into the influence of PtdChos on plasma proteome coverage, which is elaborated in the following sections. The analysis focuses on the fold changes of quantified proteins compared to plasma and the untreated corona, rather than the absolute numbers of quantified proteins. This is mainly because the numbers of quantified proteins are strongly dependent to the workflow of mass spectrometry; for example analysis of identical corona-coated polystyrene NPs by various mass spectrometry centers resulted in varying numbers of quantified proteins, ranging from 235 to more than a thousand. Therefore, this fold-change analysis is more conservative and less subject to mass spectrometry workflow biases.

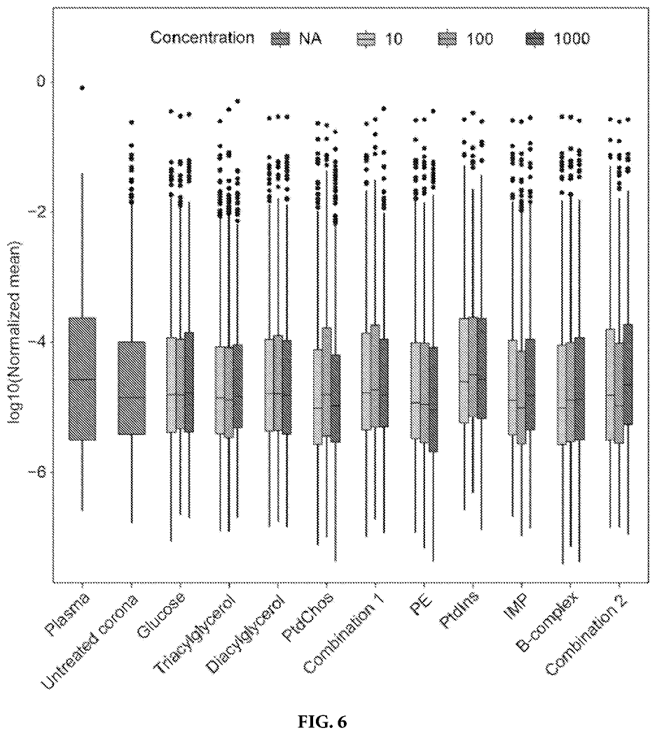

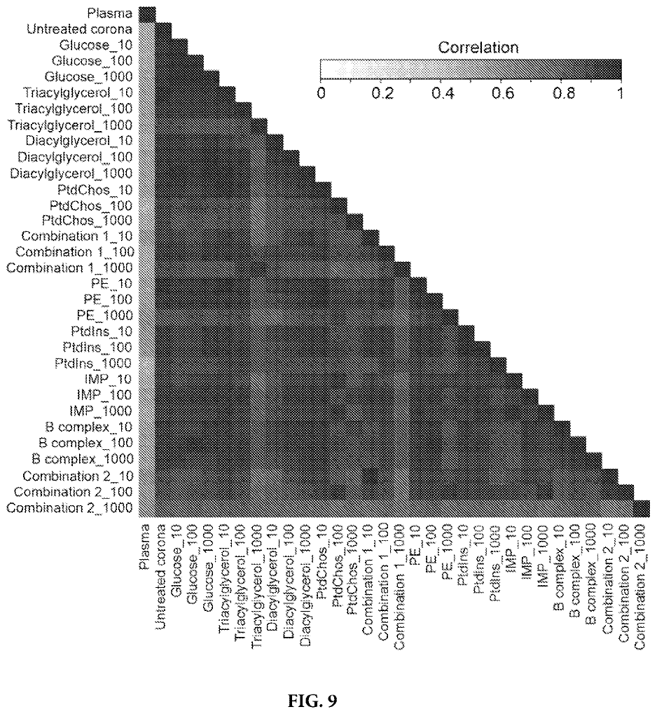

The distribution of normalized protein intensities for the samples is shown in FIG. 6. The median value in the plasma group was notably higher than in the other samples, although the overall distribution did not differ significantly. In general, the proteomes obtained from protein corona profiles in the presence of small molecules showed a good correlation (generally a Pearson correlation above 0.6 for most small molecule comparisons) demonstrating the faithful relative representation of proteins after treatment with different small molecules (FIG. 9).

### Small Molecules Diversify the Protein Corona Composition

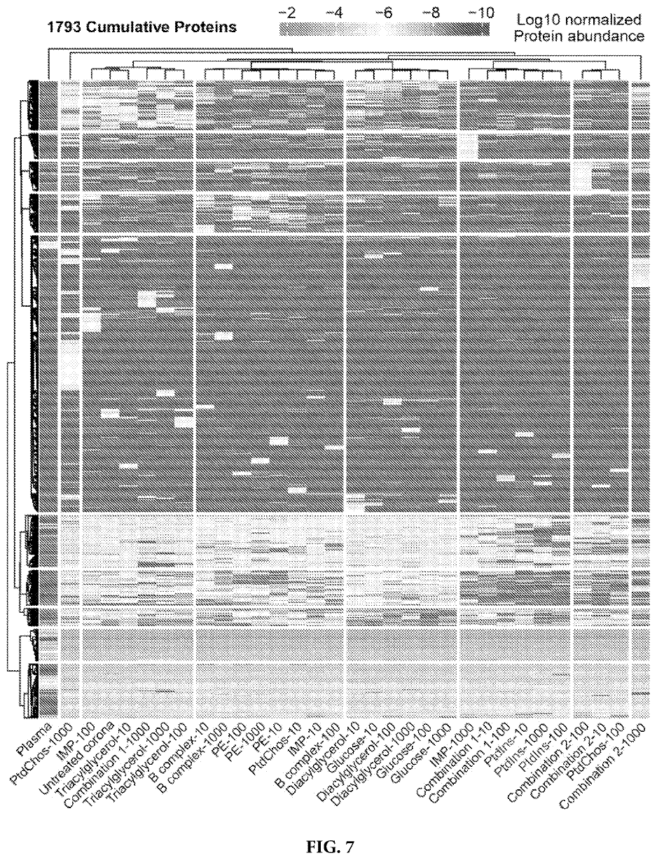

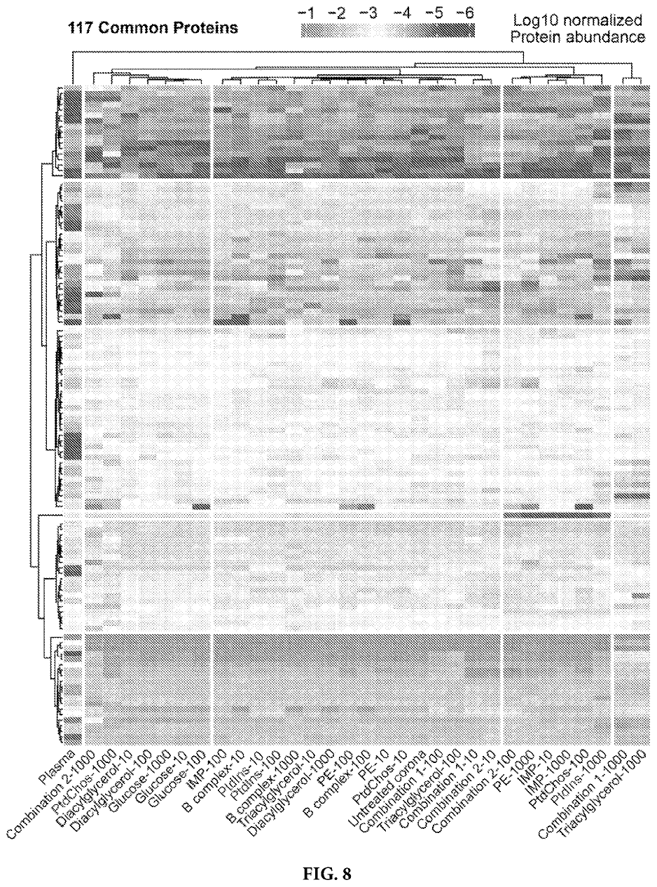

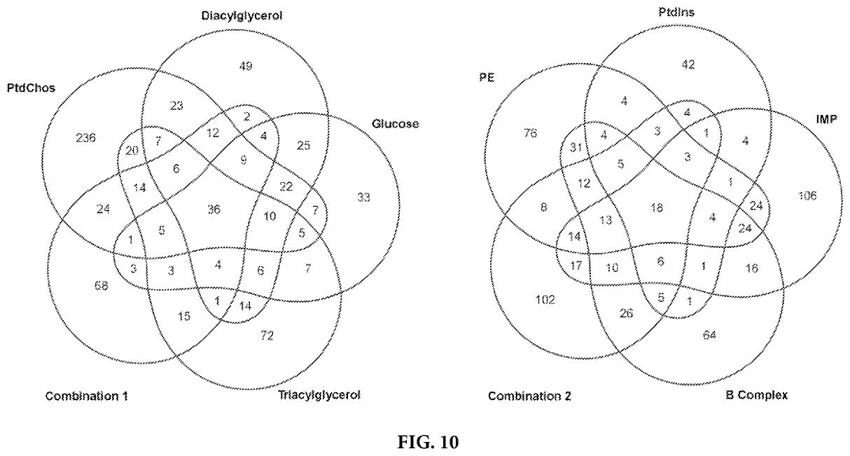

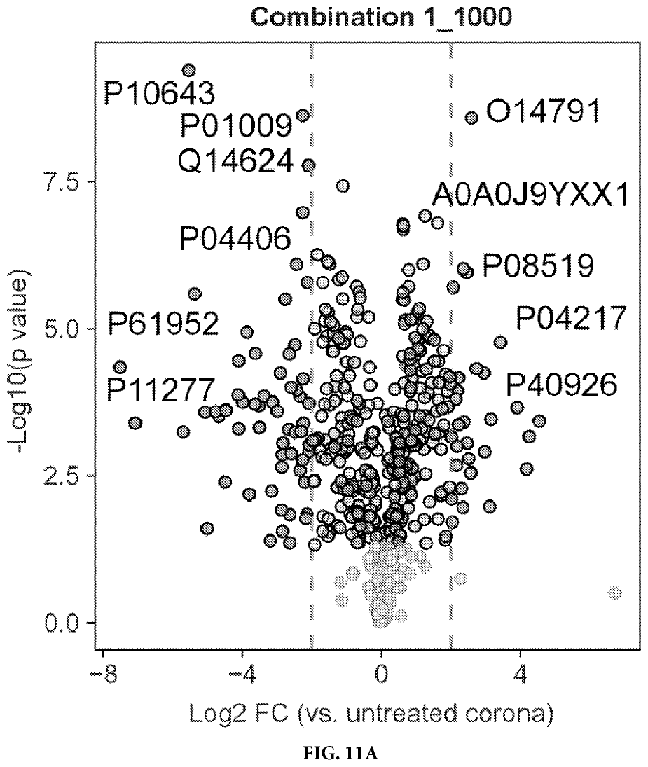

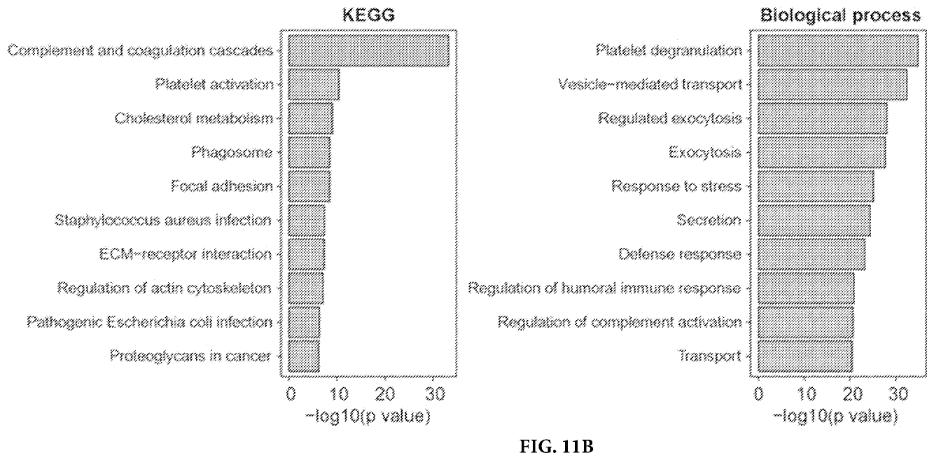

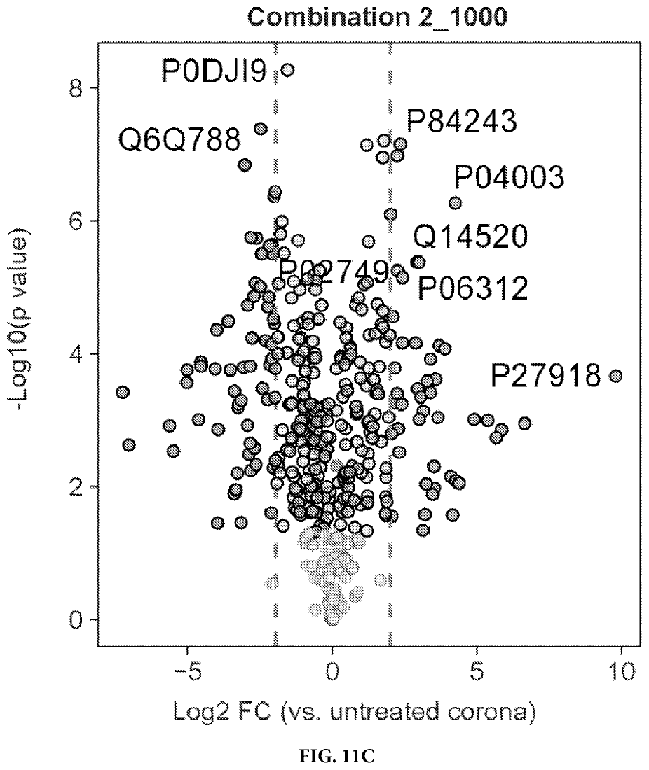

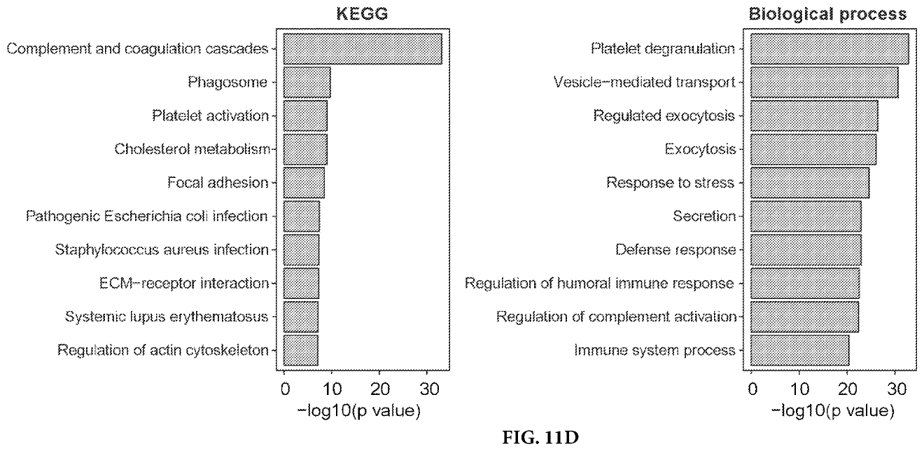

The addition of small molecules was investigated to see if they would change the type and number of proteins detected by LC-MS/MS. Indeed, each small molecule and small molecule combination generated a proteomic fingerprint that was distinct from untreated protein corona or those of other small molecules (FIG. 7). Spiking small molecules led to detection of a diverse set of proteins in the plasma. Interestingly, even different concentrations of the same small molecules or small molecule combinations produced unique fingerprints. A similar analysis was performed for the 117 shared proteins across the samples (FIG. 8). The Venn diagrams in FIG. 10 show the number of unique proteins that were quantified in the respective group across all concentrations. These results suggest that spiking small molecules into human biofluids can diversify the range of proteins that are quantifiable in protein corona profiles, effectively increasing proteomic coverage to lower abundance proteins. Such an enrichment or depletion of a specific subset of proteins can be instrumental in biomarker discovery focused on a disease area. This feature can also be used for designing assays where the enrichment of a known biomarker is facilitated by using a given small molecule. As representative examples, a comparison of enriched and depleted proteins for small molecule combinations 1 and 2 against the untreated protein corona is shown in FIG. 11A-FIG. 11B and FIG. 11C-FIG. 11D, respectively. In certain cases, the enrichment or depletion was drastic, spanning several orders of magnitude. The enriched and depleted proteins for the small molecule combinations 1 and 2 were mapped to KEGG pathways and biological processes in STRINGdb (string-db.org) (FIG. 11A and FIG. 11C). While most of the enriched pathways were shared, some pathways were specifically enriched for a given small molecule or small molecule combination. For example, systemic lupus erythematosus (SLE) was only enriched among the top pathways for small molecule combination 2 (PE, PtdIns, IMP, and vitamin B complex). Therefore, the small molecules can be potentially used for facilitating the discovery of biomarkers for specific diseases, or for assaying the abundance of a known biomarker in disease detection.

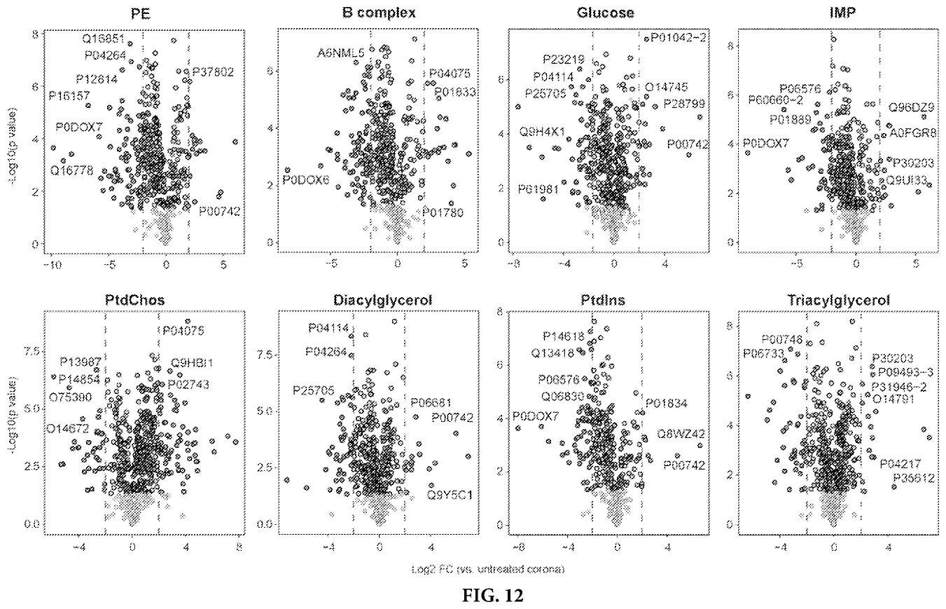

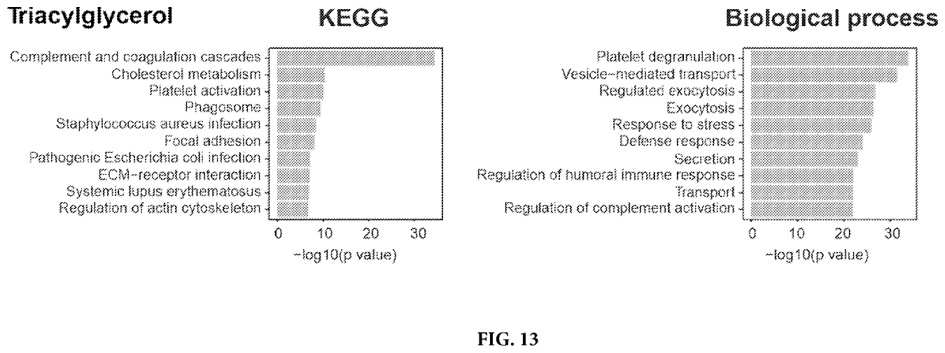

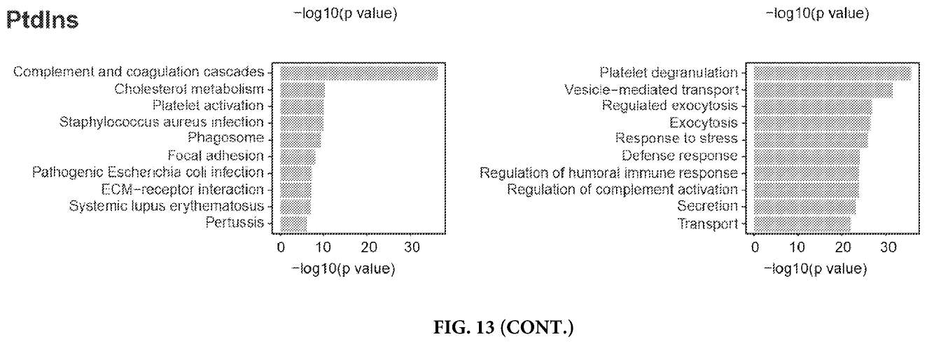

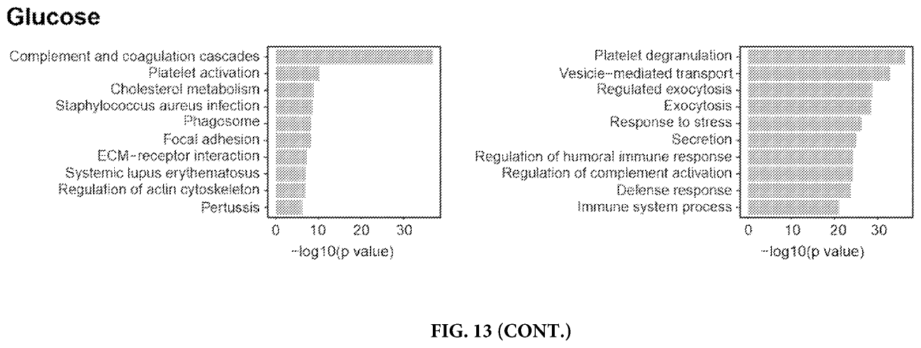

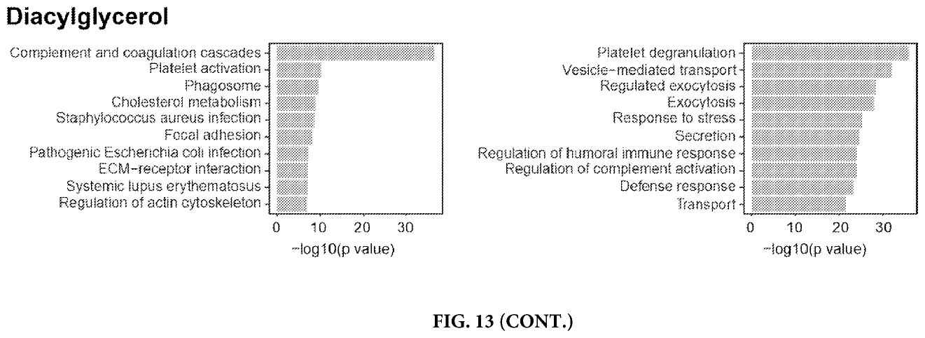

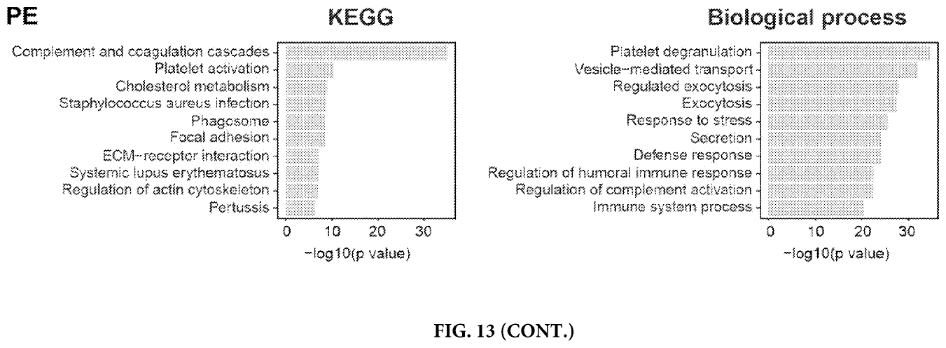

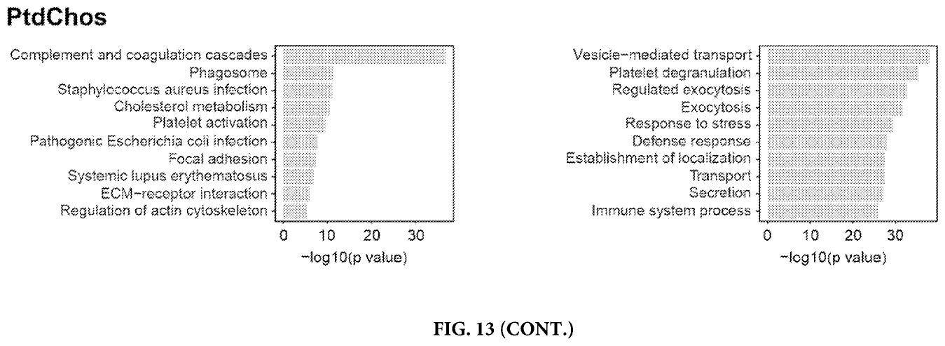

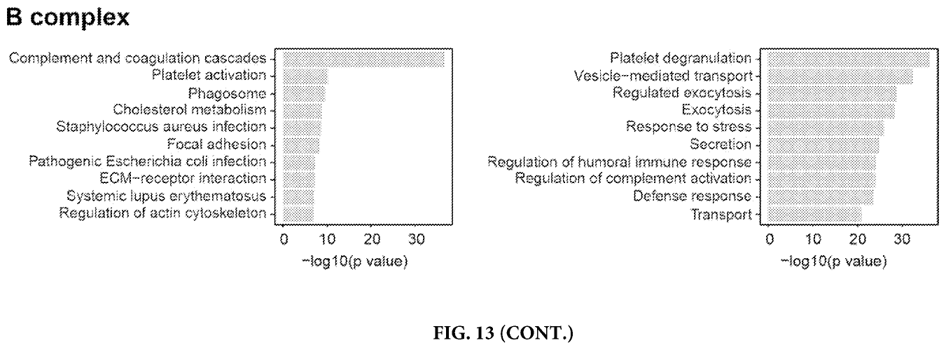

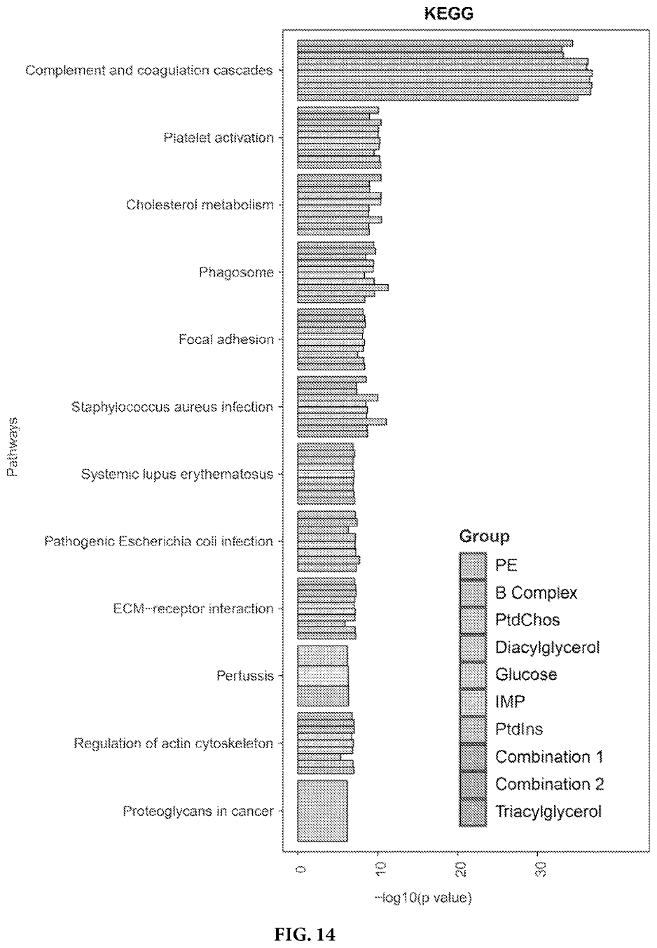

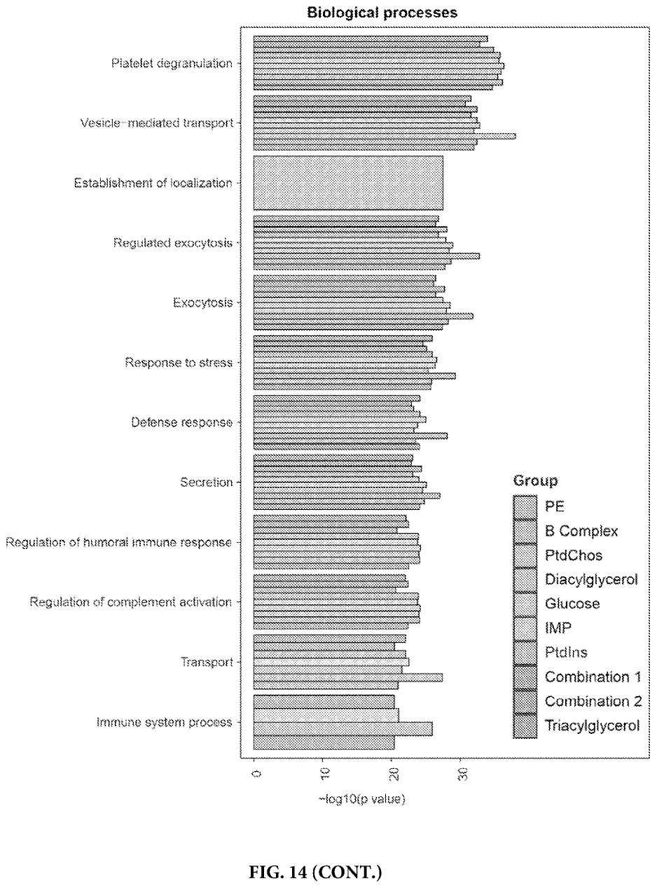

Similar analyses were performed for all the small molecules and the volcano plots for the highest concentration of each molecule (i.e., 1000 μg/ml) are demonstrated in FIG. 12. A pathway analysis was also performed for all the significantly changing proteins for each small molecule at all concentrations (FIG. 13). To facilitate comparison, the combined enrichment analysis for all the samples vs. the untreated protein corona is shown in FIG. 14.

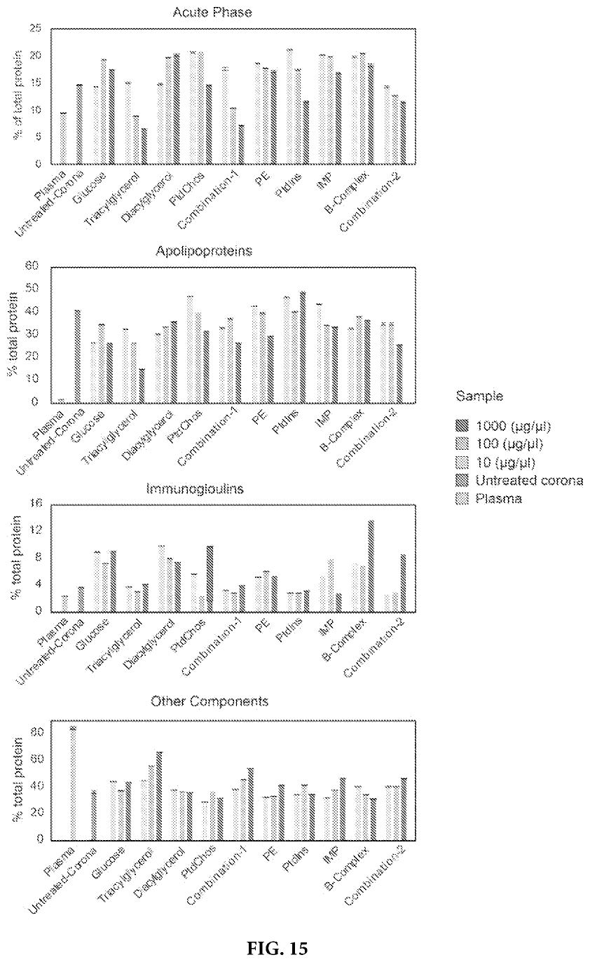

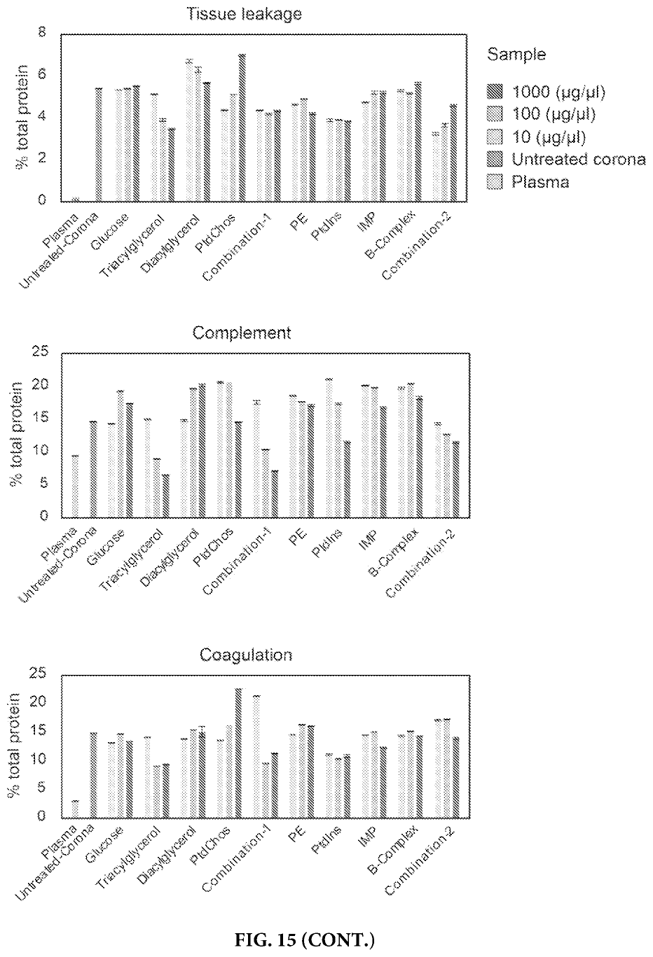

To demonstrate how small molecules affect the composition and functional categories of proteins in the protein corona, potentially aiding in early diagnosis of diseases (since proteins enriched in the corona are important in conditions like cardiovascular and neurodegenerative diseases), bioanalytical methods were used to categorize the identified proteins based on their blood-related functions, namely complement activation, immune response, coagulation, acute phase response, and lipid metabolism (FIG. 15). Different diseases may have various associations with each of the protein categories. In the analysis, apolipoproteins were major protein types that were found in the small molecule treated protein corona, and their types and abundance were heavily dependent to the type and concentrations of the employed small molecules. Similarly, the enrichment of other specific protein categories on NPs surfaces was influenced by the type and concentration of small molecules used. For example, antithrombin-III in coagulation factors plays a significant role in the protein corona composition of all tested small molecules, but this effect is observed only at their highest concentration. At lower concentrations, or in the untreated protein corona, this considerable participation is not evident. This ability of small molecules to modify the protein composition on NPs highlights their potential for early health spectrum condition and/or disease diagnosis (e.g., apolipoprotein in cardiovascular and neurodegenerative disorders), where these protein categories are crucial in disease/condition onset and progression.

### PtdChos Increases Proteome Coverage by Depleting the Abundant Plasma Proteins

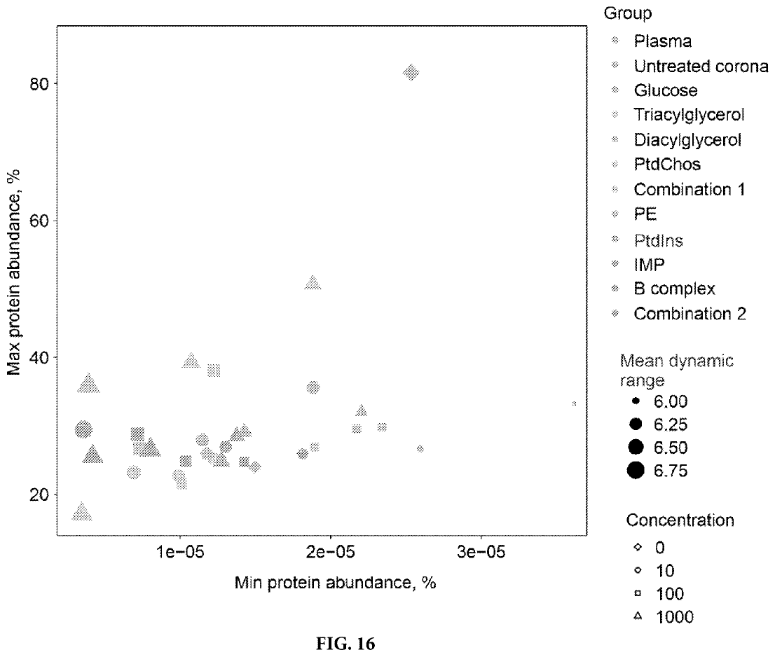

To understand whether the quantification of a higher number of proteins in protein corona profiles was due to a lower dynamic range of proteins available in human plasma for NP binding, maximum protein abundance vs. minimum protein abundance was plotted for plasma alone, and plasma treated with small molecules in FIG. 16. The plasma alone showed the highest dynamic range, suggesting that quantification of low-abundance proteins would be most difficult from plasma alone. Conversely, addition of small molecules was shown to reduce plasma protein dynamic range, thereby allowing for detection of more peptides and quantification of proteins with lower abundance through the NP protein corona.

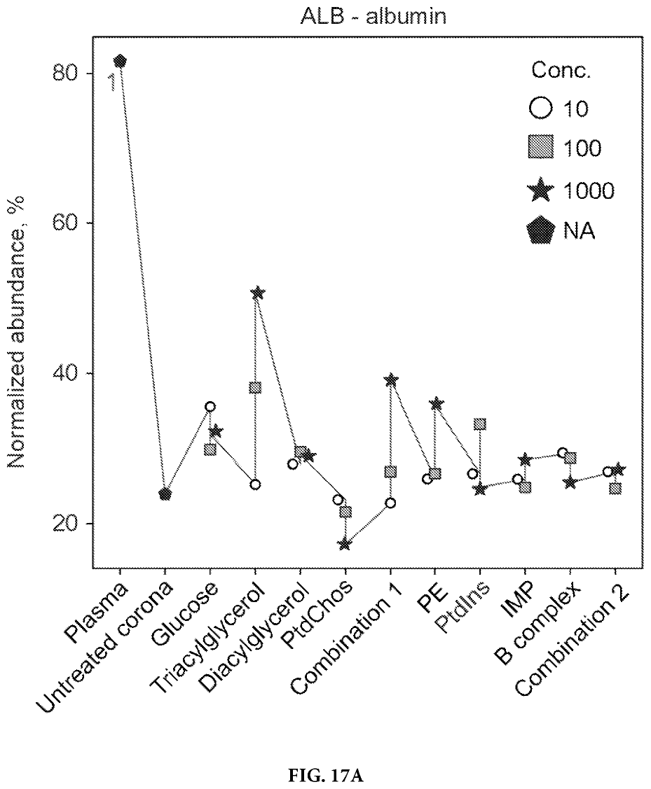

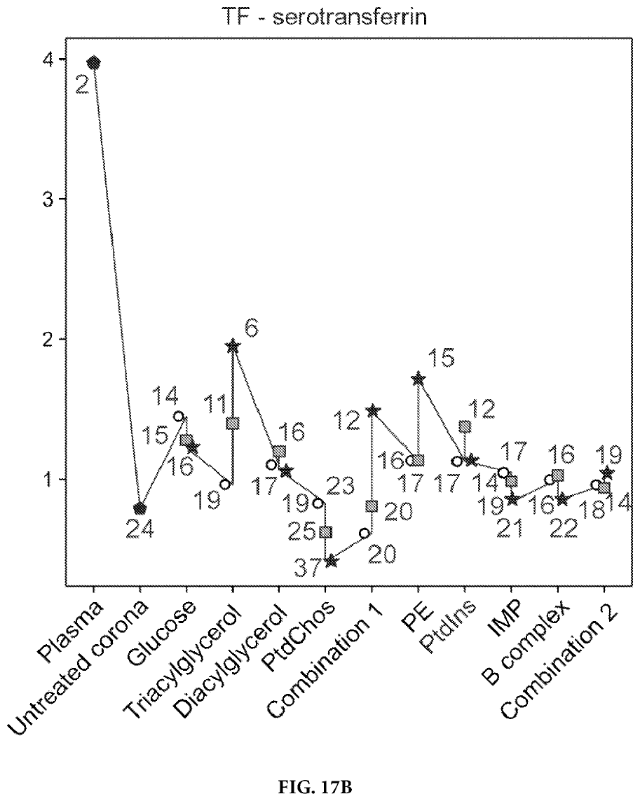

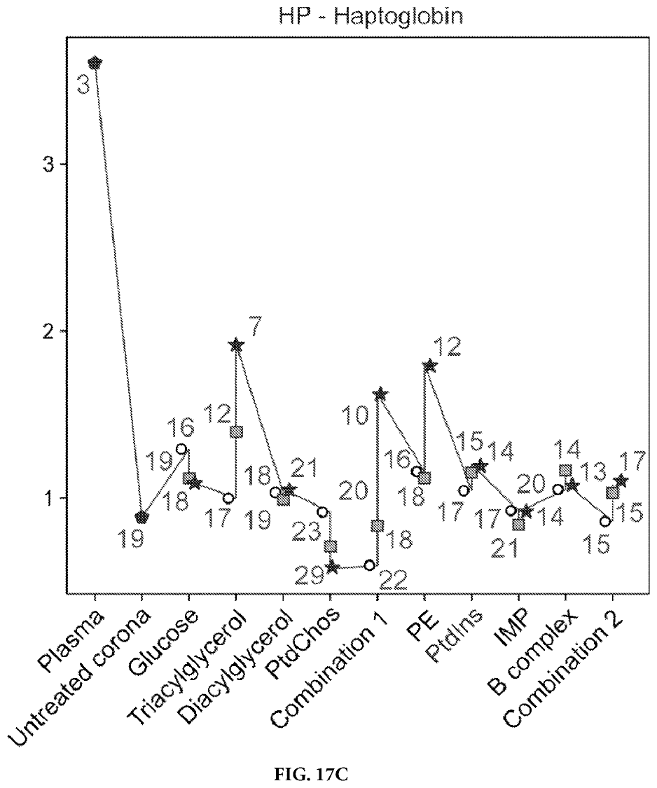

Notably, while albumin accounted for over 81% of the plasma sample, its representation was significantly lowered to an average of 29% in the protein coronas, both with and without small molecule modifications. This reduction was most pronounced with PtdChos treatment at 1000 μg/ml, where albumin levels dropped to around 17% of plasma proteins (FIG. 17A). Despite these changes, albumin remained the most abundant protein in all samples. A similar diminishing trend was observed for the second and third most abundant proteins, serotransferrin (TF) (FIG. 17B) and haptoglobin (HB) (FIG. 17C), which made up about 3.9% and 3.6% of plasma protein abundance, respectively. The rankings of these proteins' abundance in each sample are depicted above their corresponding circles in FIG. 17A-FIG. 17C. From this analysis, it is evident that the protein corona, both in its native form and when altered by small molecules, can drastically reduce the combined representation of the top three proteins from about 90% to roughly 29%. The most substantial reduction was observed with PtdChos at 1000 μg/ml, reducing the top three proteins' cumulative representation from 90% to under 17%. PtdChos treatment also effectively reduced the levels of the fourth most abundant plasma protein IGHA1.This significant decrease in the abundance of highly prevalent plasma proteins explains the marked increase in the number of unique proteins detected from NP corona samples treated with PtdChos. These results indicate that high concentrations of PtdChos can be strategically employed to enable more comprehensive plasma protein sampling by specifically targeting and depleting the most abundant plasma proteins, especially albumin.

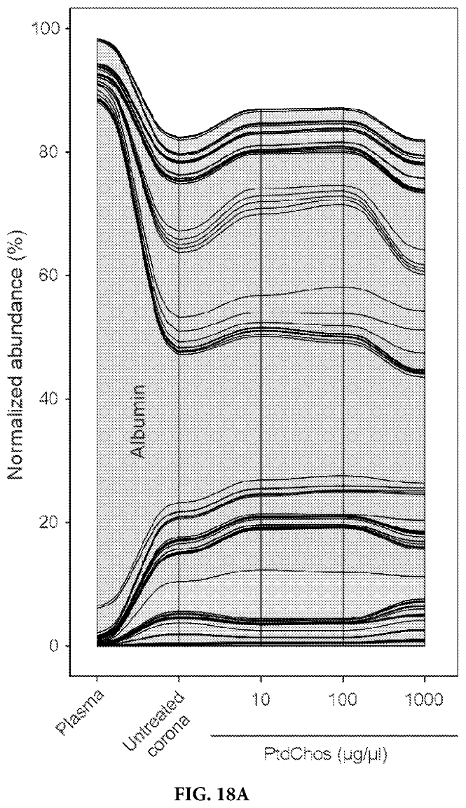

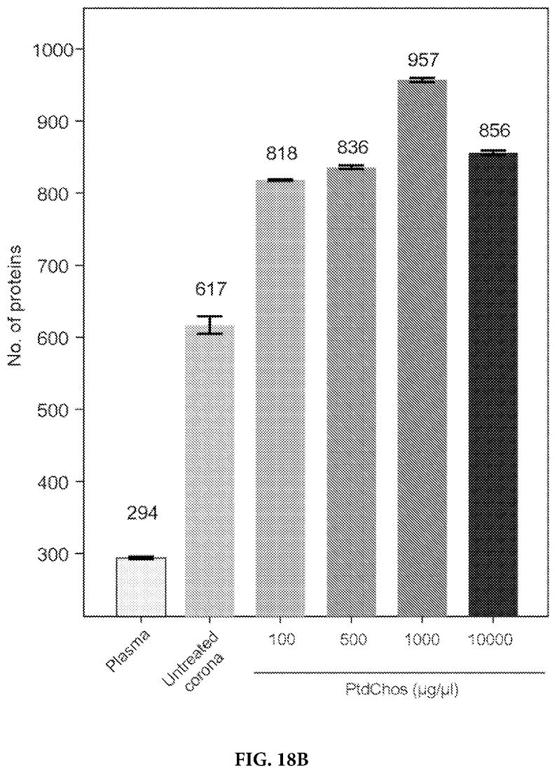

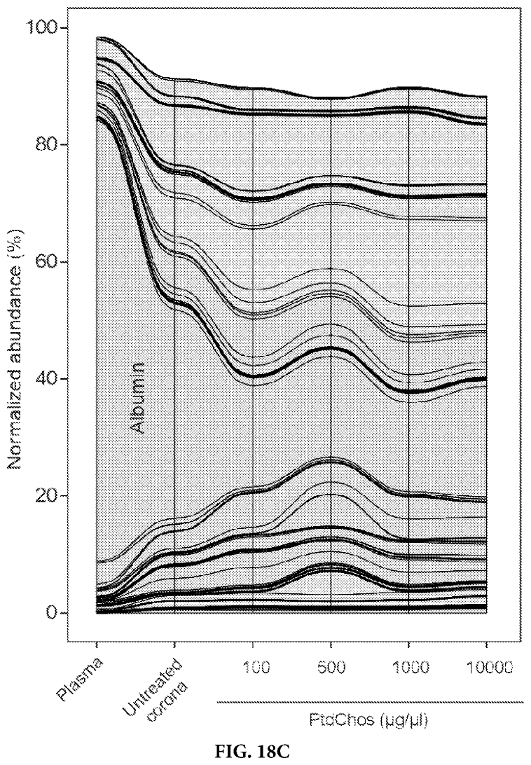

The stream (or alluvial) diagram in FIG. 18A shows the overall changes in the representation of proteins found in plasma upon incubation of protein corona with different concentrations of PtdChos. To validate this discovery, fresh samples treated with a series of PtdChos concentrations were prepared, ranging from 100 to 10,000 μg/ml. As shown in FIGS. 18B, 957 proteins could be quantified in the protein corona treated with PtdChos at 1000 μg/ml, while neither lower concentration nor further addition of PtdChos enhanced the number of quantified proteins. The stream diagram in FIG. 18D shows the specific depletion of albumin and a number of other abundant proteins in plasma upon addition of PtdChos, allowing for more robust detection of other proteins with lower abundance.

### Lc-MS Analysis by Data-Independent Acquisition (DIA)

To confirm that the improved proteome coverage achieved with PtdChos treatment is independent of the LC-MS platform used, new samples of plasma, untreated protein corona, and protein corona treated with 1000 μg/ml PtdChos were prepared and analyzed using data-independent acquisition (DIA) LC-MS.

The samples were centrifuged at 14,000xg for 20 minutes to remove the unbound or loosely bound proteins. The collected NP pellets were washed three times with cold PBS and centrifuged under the same conditions. The samples were resuspended in 20 μl of PBS, and the proteins were reduced with 2 mM dithiothreitol (DTT) (final concentration) for 45 minutes and then alkylated using 8 mM iodoacetamide (IAA) (final concentration) for 45 minutes in the dark. Subsequently, 5 μl of LysC at 0.02 μg/μl was added for 4 hours, followed by the same concentration and volume of trypsin overnight. The samples were then centrifuged at 16,000xg for 20 minutes at room temperature to remove the NPs, then cleaned using Pierce™ C 18 spin tips (Thermo Scientific™; cat number 84850) and vacuum dried.

Dried peptides were resuspended in 0.1% aqueous formic acid and subjected to LC-MS/MS analysis using an Orbitrap Exploris™ 480 Mass Spectrometer (Thermo Scientific™) fitted with a Vanquish™ Neo UHPLC System (Thermo Scientific™) and a custom-made column heater set to 60° C. Peptides were resolved using a reverse phase high-performance liquid chromatography (RP-HPLC) column (75 μM×30 cm) packed in-house with C18 resin (ReproSil®-Pur 120 C18-AQ, 1.9 μm; Dr. Maisch GmbH) at a flow rate of 0.2 μL/minute. The following gradient was used for peptide separation: from 4% buffer B to 10% buffer B over 7.5 minutes to 35% buffer B over 67.7 minutes to 50% buffer B over 15 minutes to 95% buffer B over 1 minute followed by 10 minutes at 95% buffer B to 5% buffer B over 1 minute followed by 4 minutes at 5% buffer B. Buffer A was 0.1% formic acid in water and buffer B was 80% acetonitrile (ACN), 0.1% formic acid in water.

The mass spectrometer was operated in DIA mode with a cycle time of 3 seconds. MS¹ scans were acquired in the Orbitrap Exploris™ in centroid mode at a resolution of 120,000 full-width half-medium (FWHM) (at 200 m/z), a scan range of from 390 to 910 m/z, normalized AGC target set to 300% and maximum ion injection time mode set to Auto. MS2 scans were acquired in the Orbitrap Exploris™ in centroid mode at a resolution of 15,000 FWHM (at 200 m/z), precursor mass range of 400 to 900, quadrupole isolation window of 7 m/z with 1 m/z window overlap, a defined first mass of 120 m/z, normalized AGC target set to 3000% and a maximum injection time of 22 ms. Peptides were fragmented by higher-energy collisional dissociation (HCD) with collision energy set to 28% and one microscan was acquired for each spectrum.

The acquired raw-files were searched individually using the Spectronaut® (Biognosys v18.6) directDIA workflow against a Homo sapiens database (consisting of 20,360 protein sequence downloaded from Uniprot on 2022/02/22) and 392 commonly observed contaminants. Default settings were used.

322 proteins were identified in the plasma alone, 1011 proteins in the untreated protein corona samples, and 1436 proteins in the protein corona treated with PtdChos (1.4-fold increase over the untreated corona). These findings not only validate the enhancement of plasma proteome coverage by PtdChos but also illustrate the capability of PtdChos to facilitate the in-depth profiling of the plasma proteome associated with protein corona formed on the surface of a single type of NP. Since the ratio of the number of quantified proteins through PtdChos spiking is generally around 1.4-fold higher than in the NP corona alone, PtdChos can be incorporated into any LC-MS workflow aiming to boost plasma (and any other biological sample containing albumin or highly-abundant proteins) proteome profiling. More optimized plasma proteomics pipelines or high-end mass spectrometers such as Orbitrap Astral™ (Thermo Scientific™) are envisioned to quantify an even higher number of proteins that those reported in the current study.

### Data Analysis

First, data were normalized by total protein intensity in each technical replicate. Data analysis was performed using R project version 4.1.0. Missing values were replaced by the constant value of 10\hat{ }-10 for all conditions.

### Statistics and Reproducibility

All measurements were performed as a triplicate analysis of a given aliquot. The DIA analysis was performed in one replicate.

ADDITIONAL EMBODIMENTS

Embodiment 1: A composition for detecting a number of unique/different proteins or their proteoforms in a biological sample, such as a plasma sample, the composition comprising:
(1) triacylglycerols and/or a derivative thereof;
(2) diacylglycerols, such as 1,2 and 1,3 diacylglycerol, and/or a derivative thereof;
(3) glycerophospholipids and/or a derivative thereof;
(4) a combination of triacylglycerols and/or a derivative thereof, diacylglycerols and/or a derivative thereof, and glycerophospholipids and/or a derivative thereof;
(5) glucose and/or a derivative thereof;
(6) inosine 5′-monophosphate (also known as inosinic acid or IMP) and/or a derivative thereof;
(7) vitamin B complex (also called B complex) and/or a derivative thereof;
(8) a combination of glucose and/or a derivative thereof, inosine 5′-monophosphate and/or a derivative thereof, and vitamin B complex and/or a derivative thereof;
(9) a combination of triacylglycerols and/or a derivative thereof, diacylglycerols and/or a derivative thereof; glycerophospholipids and/or derivative thereof, glucose and/or a derivative thereof, inosine 5′-monophosphate and/or a derivative thereof, and vitamin B complex and/or a derivative thereof;
(10) phosphatidylcholine and/or a derivative thereof;
(11) phosphatidylethanolamine and/or a derivative thereof;
(12) phosphatidylserine and/or a derivative thereof;
(13) phosphatidic acid and/or a derivative thereof;
(14) phosphatidylinositol and/or a derivative thereof;
(15) phosphatidylglycerol and/or a derivative thereof;
(16) cardiolipin and/or a derivative thereof;
(17) L-α-phosphatidylinositol and/or a derivative thereof;
(18) a combination of glucose and/or a derivative thereof, triacylglycerols and/or a derivative thereof, diacylglycerols and/or a derivative thereof; and phosphatidylcholine and/or a derivative thereof;
(19) a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof; or
(20) a combination of any one of (1) to (19).

Embodiment 2: The composition of embodiment 1 further comprising one or more protein-binding agent.

Embodiment 3: The composition of embodiment 1 or 2 further comprising one or more biological sample.

Embodiment 4: The composition of any one of embodiments 1-3, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 5: The composition of any one of embodiments 1-4, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 6: The composition of any one of embodiments 1-5, wherein the one or more protein-binding agent has a polydispersity index (PDI) of about 0.01 to about 0.7, preferably about 0.3.

Embodiment 7: The composition of any one of embodiments 1-6, wherein the protein-binding agents are of the same type.

Embodiment 8: The composition of any one of embodiments 1-7, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 9: The composition of any one of embodiments 1-8, wherein the one or more small molecule is phosphatidylcholine.

Embodiment 10: The composition of embodiment 9, wherein about 1 pg/ml to about 1 g/ml phosphatidylcholine is added to the biological sample, such as about 1000 μg/ml.

Embodiment 11: The composition of any one of embodiments 1-10, wherein the one or more small molecule is added to the biological sample at diverse concentrations.

Embodiment 12: The composition of embodiment 11, wherein the diverse concentrations are from about 1 pg/ml to about 1 g/ml.

Embodiment 13: A method for detecting proteins and/or their proteoforms in a biological sample, the method comprising:
adding one or more small molecule to the biological sample;
adding one or more protein-binding agent having a surface capable of binding proteins to the biological sample and allowing a protein corona to form on the surface of the one or more protein-binding agent to produce a complex comprising the protein corona and the one more protein-binding agent; and
detecting the number of proteins and/or their proteoforms in the protein corona by an antibody-based technique or a proteomics technique.

Embodiment 14: The method of embodiment 13, wherein detecting proteins and/or their proteoforms in the protein corona is by a proteomics technique comprising top-down, middle-down or bottom-up LC-MS/MS.

Embodiment 15: The method of embodiment 13 or 14, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 16: The method of any one of embodiments 13-15, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 17: The method of any one of embodiments 13-16, wherein the one or more protein-binding agent has a polydispersity index (PDI) of about 0.01 to about 0.7, preferably about 0.3.

Embodiment 18: The method of any one of embodiments 13-17, wherein the protein-binding agents are of the same type.

Embodiment 19: The method of any one of embodiments 13-18, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 20: The method of any one of embodiments 13-19, wherein the one or more small molecule comprises a biomolecule.

Embodiment 21: The method of any one of embodiments 13-20, wherein the one or more small molecule comprises a metabolite, a lipid, a nutrient, a plant-derived molecule, or a combination thereof.

Embodiment 22: The method of embodiment 21, wherein the one or more small molecule comprises:
a triacylglycerol and/or a derivative thereof;
a diacylglycerol, such as 1,2 and 1,3 diacylglycerol, and/or a derivative thereof;
a glycerophospholipid and/or a derivative thereof;
a combination of a triacylglycerol and/or a derivative thereof, a diacylglycerol and/or a derivative thereof, and a glycerophospholipid and/or a derivative thereof;
glucose and/or a derivative thereof;
inosine 5′-monophosphate and/or a derivative thereof;
vitamin B complex and/or a derivative thereof;
a combination of glucose and/or a derivative thereof, inosine 5′-monophosphate and/or a derivative thereof, and vitamin B complex and/or a derivative thereof;
a combination of a triacylglycerol and/or a derivative thereof, a diacylglycerol and/or a derivative thereof; a glycerophospholipid and/or derivative thereof, glucose and/or a derivative thereof, inosine 5′-monophosphate and/or a derivative thereof, and vitamin B complex and/or a derivative thereof;
phosphatidylcholine and/or a derivative thereof;
phosphatidylethanolamine and/or a derivative thereof;
phosphatidylserine and/or a derivative thereof;
phosphatidic acid and/or a derivative thereof;
phosphatidylinositol and/or a derivative thereof;
phosphatidylglycerol and/or a derivative thereof;
cardiolipin and/or a derivative thereof;
L-α-phosphatidylinositol and/or a derivative thereof;
a combination of glucose and/or a derivative thereof, a triacylglycerol and/or a derivative thereof, a diacylglycerol and/or a derivative thereof; and phosphatidylcholine and/or a derivative thereof;
a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof; or
a combination thereof.

Embodiment 23: The method of any one of embodiments 13-22,wherein the one or more small molecule is phosphatidylcholine.

Embodiment 24: The method of embodiment 23, wherein about 1 pg/ml to about 1 g/ml phosphatidylcholine is added to the biological sample, such as about 1000 μg/ml.

Embodiment 25: The method of any one of embodiments 13-24, wherein the biological sample is run through a depletion column or a spin column with resin.

Embodiment 26: The method of any one of embodiments 13-25, wherein
at least a 0.1-fold, 0.5-fold, 1-fold, or more increase in the number of proteins and/or their proteoforms is observed compared to a biological sample without the one or more protein-binding agent and the one or more small molecule; and/or
at least a 0.1-fold, 0.5-fold, 1-fold, or more increase in the number of proteins and/or their proteoforms compared to a biological sample comprising the one or more protein-binding agent without the addition of the one or more small molecule.

Embodiment 27: The method of any one of embodiments 13-26, wherein one or more small molecule is added to the biological sample at diverse concentrations.

Embodiment 28: The method of embodiment 27, wherein the diverse concentrations are from about 1 pg/ml to about 1 g/ml.

Embodiment 29: The method of any one of embodiments 13-28, further comprising preparing the complex for detecting the proteins and/or their proteoforms in the protein corona.

Embodiment 30: The method of embodiment 29 comprising:
separating the complex from the remainder of the biological sample;
washing the complex;
resuspending the complex;
reducing the proteins from the complex;
alkylating the proteins from the complex; and
digesting the proteins from the complex.

Embodiment 31: The method of any one of embodiments 13-30, wherein the one or more small molecule interacts with one or more unique/different proteins or their proteoforms in the biological sample.

Embodiment 32: The method of any one of embodiments 13-31, wherein the one or more small molecule enhances detection of low-abundance proteins.

Embodiment 33: The method of embodiment 23, wherein phosphatidylcholine binds albumin.

Embodiment 34: The method of any one of embodiments 13-33, wherein about 1 pg/ml to about 1 g/ml of the one or more protein-binding agent are added to the biological sample.

Embodiment 35: A method for detecting one or more biomarker or a pattern of one or more biomarker associated with a disease, the method comprising:
adding one or more small molecule to at least two biological samples each biological sample from different subjects diagnosed with the disease;
adding one or more protein-binding agent having a surface capable of binding proteins to the biological samples and allowing a protein corona to form on the surface of the one or more protein-binding agent to produce a complex comprising the protein corona and the one or more protein-binding agent; and
detecting the one or more biomarker or the pattern of one or more biomarker in the protein corona by an antibody-based technique or a proteomics technique.

Embodiment 36: The method of embodiment 35, wherein detecting the one or more biomarker or the pattern of one or more biomarker in the protein corona is by a proteomics technique comprising top-down, middle-down or bottom-up LC-MS/MS.

Embodiment 37: The method of embodiment 35 or 36, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 38: The method of any one of embodiments 35-37, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 39: The method of any one of embodiments 35-38, wherein the one or more protein-binding agent has a polydispersity index (PDI) of about 0.01 to about 0.7, preferably about 0.3.

Embodiment 40: The method of any one of embodiments 35-39, wherein the protein-binding agents are of the same type.

Embodiment 41: The method of any one of embodiments 35-40, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 42: The method of any one of embodiments 35-41, wherein the one or more small molecule comprises a biomolecule.

Embodiment 43: The method of any one of embodiments 35-42, wherein the one or more small molecule comprises a metabolite, a lipid, a nutrient, a plant-derived molecule, or a combination thereof.

Embodiment 44: The method of embodiment 43, wherein the one or more small molecule comprises:
a triacylglycerol and/or a derivative thereof;
a diacylglycerol, such as 1,2 and 1,3 diacylglycerol, and/or a derivative thereof;
a glycerophospholipid and/or a derivative thereof;
a combination of a triacylglycerol and/or a derivative thereof, a diacylglycerol and/or a derivative thereof, and a glycerophospholipid and/or a derivative thereof;
glucose and/or a derivative thereof;
inosine 5′-monophosphate and/or a derivative thereof;
vitamin B complex and/or a derivative thereof;
a combination of glucose and/or a derivative thereof, inosine 5′-monophosphate and/or a derivative thereof, and vitamin B complex and/or a derivative thereof;
a combination of a triacylglycerol and/or a derivative thereof, a diacylglycerol and/or a derivative thereof; a glycerophospholipid and/or derivative thereof, glucose and/or a derivative thereof, inosine 5′-monophosphate and/or a derivative thereof, and vitamin B complex and/or a derivative thereof;
phosphatidylcholine and/or a derivative thereof;
phosphatidylethanolamine and/or a derivative thereof;
phosphatidylserine and/or a derivative thereof;
phosphatidic acid and/or a derivative thereof;
phosphatidylinositol and/or a derivative thereof;
phosphatidylglycerol and/or a derivative thereof;
cardiolipin and/or a derivative thereof;
L-60 -phosphatidylinositol and/or a derivative thereof;
a combination of glucose and/or a derivative thereof, a triacylglycerol and/or a derivative thereof, a diacylglycerol and/or a derivative thereof; and phosphatidylcholine and/or a derivative thereof;
a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof; or
a combination thereof.

Embodiment 45: The method of any one of embodiments 35-44, wherein the one or more small molecule is phosphatidylcholine.

Embodiment 46: The method of embodiment 45, wherein about 1 pg/ml to about 1 g/ml phosphatidylcholine is added to the biological sample, such as about 1000 μg/ml.

Embodiment 47: The method of any one of embodiments 35-46, wherein the biological sample is run through a depletion column or a spin column with resin.

Embodiment 48: The method of any one of embodiments 35-47, wherein
at least a 0.1-fold, 0.5-fold, 1-fold, or more increase in the number of biomarkers or patterns of biomarkers is observed compared to a biological sample without the one or more protein-binding agent and the one or more small molecule; and/or
at least a 0.1-fold, 0.5-fold, 1-fold, or more increase in the number of biomarkers or patterns of biomarkers compared to a biological sample comprising the one or more protein-binding agent without the addition of the one or more small molecule.

Embodiment 49: The method of any one of embodiments 35-48, wherein one or more small molecule is added to the biological sample at diverse concentrations.

Embodiment 50: The method of embodiment 49, wherein the diverse concentrations are from about 1 pg/ml to about 1 g/ml.

Embodiment 51: The method of any one of embodiments 35-50, further comprising preparing the complex for detecting the or more biomarker or a pattern of one or more biomarker associated with a disease in the protein corona.

Embodiment 52: The method of embodiment 51 comprising:
separating the complex from the remainder of the biological sample;
washing the complex;
resuspending the complex;
reducing the proteins from the complex;
alkylating the proteins from the complex; and
digesting the proteins from the complex.

Embodiment 53: The method of any one of embodiments 35-52, wherein the disease is a neoplastic disease, cardiovascular disease, metabolic disease, infectious disease, inflammatory disease, congenital and hereditary diseases, degenerative disease, a neurological disease, or a combination thereof.

Embodiment 54: The method of embodiment 53, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.

Embodiment 55: The method of embodiment 53, wherein the disease is a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

Embodiment 56: The method of any one of embodiments 35-55, wherein the one or more small molecule interacts with one or more unique/different proteins in the biological sample.

Embodiment 57: The method of any one of embodiments 35-56, wherein the one or more small molecule enhances detection of low-abundance proteins.

Embodiment 58: The method of embodiment 45, wherein phosphatidylcholine binds albumin.

Embodiment 59: The method of any one of embodiments 35-58, wherein about 1 pg/ml to about 1 g/ml of protein-binding agents are added to the biological sample.

Embodiment 60: A method of diagnosing a disease in a subject, the method comprising:
adding one or more small molecule to a biological sample from the subject;
adding one or more protein-binding agent having a surface capable of binding proteins to the biological sample and allowing a protein corona to form on the surface of the one or more protein-binding agent to produce a complex comprising the protein corona and the one or more protein-binding agent; and
detecting one or more biomarker or a pattern of one or more biomarker associated with the disease in the protein corona by an antibody-based technique or a proteomics technique.

Embodiment 61: The method of embodiment 60, wherein detecting the one or more biomarker or the pattern of one or more biomarker in the protein corona is by a proteomics technique comprising top-down, middle-down or bottom-up LC-MS/MS.

Embodiment 62: The method of embodiment 60 or 61, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 63: The method of any one of embodiments 60-62, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 64: The method of any one of embodiments 60-63, wherein the one or more protein-binding agent has a polydispersity index (PDI) of about 0.01 to about 0.7, preferably about 0.3.

Embodiment 65: The method of any one of embodiments 60-64, wherein the protein-binding agents are of the same type.

Embodiment 19: The method of any one of embodiments 13-18, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 66: The method of any one of embodiments 60-65, wherein the one or more small molecule comprises a biomolecule.

Embodiment 67: The method of any one of embodiments 60-66, wherein the one or more small molecule comprises a metabolite, a lipid, a nutrient, a plant-derived molecule, or a combination thereof.

Embodiment 68: The method of embodiment 67, wherein the one or more small molecule comprises:
a triacylglycerol and/or a derivative thereof;
a diacylglycerol, such as 1,2 and 1,3 diacylglycerol, and/or a derivative thereof;
a glycerophospholipid and/or a derivative thereof;
a combination of a triacylglycerol and/or a derivative thereof, a diacylglycerol and/or a derivative thereof, and a glycerophospholipid and/or a derivative thereof;
glucose and/or a derivative thereof;
inosine 5′-monophosphate and/or a derivative thereof;
vitamin B complex and/or a derivative thereof;
a combination of glucose and/or a derivative thereof, inosine 5′-monophosphate and/or a derivative thereof, and vitamin B complex and/or a derivative thereof;
a combination of a triacylglycerol and/or a derivative thereof, a diacylglycerol and/or a derivative thereof; a glycerophospholipid and/or derivative thereof, glucose and/or a derivative thereof, inosine 5′-monophosphate and/or a derivative thereof, and vitamin B complex and/or a derivative thereof;
phosphatidylcholine and/or a derivative thereof;
phosphatidylethanolamine and/or a derivative thereof;
phosphatidylserine and/or a derivative thereof;
phosphatidic acid and/or a derivative thereof;
phosphatidylinositol and/or a derivative thereof;
phosphatidylglycerol and/or a derivative thereof;
cardiolipin and/or a derivative thereof;
L-α-phosphatidylinositol and/or a derivative thereof;
a combination of glucose and/or a derivative thereof, a triacylglycerol and/or a derivative thereof, a diacylglycerol and/or a derivative thereof; and phosphatidylcholine and/or a derivative thereof;
a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof; or
a combination thereof.

Embodiment 69: The method of any one of embodiments 60-68, wherein the one or more small molecule is phosphatidylcholine.

Embodiment 70: The method of embodiment 69, wherein about 1 pg/ml to about 1 g/ml phosphatidylcholine is added to the biological sample, such as about 1000 μg/ml.

Embodiment 71: The method of any one of embodiments 60-70, wherein the biological sample is run through a depletion column or a spin column with resin.

Embodiment 72: The method of any one of embodiments 60-71, wherein
at least a 0.1-fold, 0.5-fold, 1-fold, or more increase in the number of biomarkers or patterns of biomarkers is observed compared to a biological sample without the one or more protein-binding agent and the one or more small molecule; and/or
at least a 0.1-fold, 0.5-fold, 1-fold, or more increase in the number of biomarkers or patterns of biomarkers compared to a biological sample comprising the one or more protein-binding agent without the addition of the one or more small molecule.

Embodiment 73: The method of any one of embodiments 60-72, wherein one or more small molecule is added to the biological sample at diverse concentrations.

Embodiment 74: The method of embodiment 73, wherein the diverse concentrations are from about 1 pg/ml to about 1 g/ml.

Embodiment 75: The method of any one of embodiments 60-74, further comprising preparing the complex for detecting the one or more biomarker or a pattern of one or more biomarker associated with the disease in the protein corona.

Embodiment 76: The method of embodiment 75 comprising:
separating the complex from the remainder of the biological sample;
washing the complex;
resuspending the complex;
reducing the proteins from the complex;
alkylating the proteins from the complex; and
digesting the proteins from the complex.

Embodiment 77: The method of any one of embodiments 60-76, wherein the disease is a neoplastic disease, cardiovascular disease, metabolic disease, infectious disease, inflammatory disease, congenital and hereditary diseases, degenerative disease, a neurological disease, or a combination thereof.
Embodiment 78: The method of embodiment 77, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.
Embodiment 79: The method of embodiment 77, wherein the disease is a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

Embodiment 80: The method of any one of embodiments 60-79, wherein the one or more small molecule interacts with one or more unique/different proteins in the biological sample.

Embodiment 81: The method of any one of embodiments 60-80, wherein the one or more small molecule enhances detection of low-abundance proteins.

Embodiment 82: The method of embodiment 69, wherein phosphatidylcholine binds albumin.

Embodiment 83: The method of any one of embodiments 60-82, wherein about 1 pg/ml to about 1 g/ml of protein-binding agents are added to the biological sample.

Embodiment 84: A method for detecting proteins and/or their proteoforms in a biological sample, the method comprising:
adding a means for binding one or more high-abundance protein and/or their proteoforms in the biological sample;
adding one or more protein-binding agent having a surface capable of binding proteins to the biological sample and allowing a protein corona to form on the surface of the one or more protein-binding agent to produce a complex comprising the protein corona and the one more protein-binding agent; and
detecting the number of proteins and/or their proteoforms in the protein corona by an antibody-based technique or a proteomics technique.

Embodiment 85: The method of embodiment 84, wherein the high-abundance protein is albumin.

Embodiment 86: The method of embodiment 84 or 85, wherein detecting high-abundance proteins and/or their proteoforms in the protein corona is by a proteomics technique comprising top-down, middle-down or bottom-up LC-MS/MS.

Embodiment 87: The method of any one of embodiments 84-86, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 88: The method of any one of embodiments 84-87, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 89: The method of any one of embodiments 84-88, wherein the one or more protein-binding agent has a polydispersity index (PDI) of about 0.01 to about 0.7, preferably about 0.3.

Embodiment 90: The method of any one of embodiments 84-89, wherein when there is more than one protein-binding agent, the protein-binding agents are of the same type.

Embodiment 91: The method of any one of embodiments 84-90, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 92: The method of any one of embodiments 84-91, wherein the biological sample is run through a depletion column or a spin column with resin.

Embodiment 93: The method of any one of embodiments 84-92, wherein
at least a 0.1-fold, 0.5-fold, 1-fold, or more increase in the number of proteins and/or their proteoforms is observed compared to a biological sample without the one or more protein-binding agent and the means for binding one or more high-abundance protein and/or their proteoforms; and/or
at least a 0.1-fold, 0.5-fold, 1-fold, or more increase in the number of proteins and/or their proteoforms compared to a biological sample comprising the one or more protein-binding agent without the addition of the means for binding one or more high-abundance protein and/or their proteoforms.

Embodiment 94: The method of any one of embodiments 84-93, further comprising preparing the complex for detecting the proteins and/or their proteoforms in the protein corona.

Embodiment 95: The method of embodiment 94 comprising:
separating the complex from the remainder of the biological sample;
washing the complex;
resuspending the complex;
reducing the proteins from the complex;
alkylating the proteins from the complex; and
digesting the proteins from the complex.

Embodiment 96: The method of any one of embodiments 84-30, wherein about 1 pg/ml to about 1 g/ml of protein-binding agents are added to the biological sample.

Embodiment 97: A method for detecting one or more biomarker or a pattern of one or more biomarker associated with a disease, the method comprising:
adding a means for binding one or more high-abundance protein and/or their proteoforms in at least two biological samples each biological sample from different subjects diagnosed with the disease;
adding one or more protein-binding agent having a surface capable of binding proteins to the biological samples and allowing a protein corona to form on the surface of the one or more protein-binding agent to produce a complex comprising the protein corona and the one or more protein-binding agent; and
detecting the one or more biomarker or the pattern of one or more biomarker in the protein corona by an antibody-based technique or a proteomics technique.

Embodiment 98: The method of embodiment 97, wherein the high-abundance protein is albumin.

Embodiment 99: The method of embodiment 97 or 98, wherein detecting the one or more biomarker or the pattern of one or more biomarker in the protein corona is by a proteomics technique comprising top-down, middle-down or bottom-up LC-MS/MS.

Embodiment 100: The method of any one of embodiments97-99, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 101: The method of any one of embodiments 97-100, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 102: The method of any one of embodiments 97-202, wherein the one or more protein-binding agent has a polydispersity index (PDI) of about 0.01 to about 0.7, preferably about 0.3.

Embodiment 103: The method of any one of embodiments 97-102, wherein when there is more than one protein-binding agent, the protein-binding agents are of the same type.

Embodiment 104: The method of any one of embodiments 97-103, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 105: The method of any one of embodiments 97-104, wherein the biological sample is run through a depletion column or a spin column with resin.

Embodiment 106: The method of any one of embodiments 97-105, wherein
at least a 0.1-fold, 0.5-fold, 1-fold, or more increase in the number of biomarkers or patterns of biomarkers is observed compared to a biological sample without the one or more protein-binding agent and the means for binding one or more high-abundance protein and/or their proteoforms; and/or
at least a 0.1-fold, 0.5-fold, 1-fold, or more increase in the number of biomarkers or patterns of biomarkers compared to a biological sample comprising the one or more protein-binding agent without the addition of the means for binding one or more high-abundance protein and/or their proteoforms.

Embodiment 107: The method of any one of embodiments 97-106, further comprising preparing the complex for detecting the or more biomarker or a pattern of one or more biomarker associated with a disease in the protein corona.

Embodiment 108: The method of embodiment 107 comprising:
separating the complex from the remainder of the biological sample;
washing the complex;
resuspending the complex;
reducing the proteins from the complex;
alkylating the proteins from the complex; and
digesting the proteins from the complex.

Embodiment 109: The method of any one of embodiments 97-108, wherein the disease is a neoplastic disease, cardiovascular disease, metabolic disease, infectious disease, inflammatory disease, congenital and hereditary diseases, degenerative disease, a neurological disease, or a combination thereof.

Embodiment 110: The method of embodiment 109, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.

Embodiment 111: The method of embodiment 109, wherein the disease is a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

Embodiment 112: The method of any one of embodiments 97-111, wherein about 1 pg/ml to about 1 g/ml of protein-binding agents are added to the biological sample.

Embodiment 113: A method of diagnosing a disease in a subject, the method comprising:
adding a means for binding one or more high-abundance protein and/or their proteoforms in a biological sample from the subject;
adding one or more protein-binding agent having a surface capable of binding proteins to the biological sample and allowing a protein corona to form on the surface of the one or more protein-binding agent to produce a complex comprising the protein corona and the one or more protein-binding agent; and
detecting one or more biomarker or a pattern of one or more biomarker associated with the disease in the protein corona by an antibody-based technique or a proteomics technique.

Embodiment 114: The method of embodiment 113, wherein the high-abundance protein is albumin.

Embodiment 115: The method of embodiment 113 or 114, wherein detecting the one or more biomarker or the pattern of one or more biomarker in the protein corona is by a proteomics technique comprising top-down, middle-down or bottom-up LC-MS/MS.

Embodiment 116: The method of any one of embodiments 113-115, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

Embodiment 117: The method of any one of embodiments 113-116, wherein the one or more protein-binding agent comprises one or more nanoscale or microscale material, such as a nanoparticle, nanorod, nanosphere, nanodisk, nanocluster, nanofiber, nanotube, microparticle, microrod, microsphere, microbead, or a combination thereof.

Embodiment 118: The method of any one of embodiments 113-117, wherein the one or more protein-binding agent has a polydispersity index (PDI) of about 0.01 to about 0.7, preferably about 0.3.

Embodiment 119: The method of any one of embodiments 113-118, wherein when there is more than one protein-binding agent, the protein-binding agents are of the same type.

Embodiment 120: The method of any one of embodiments 113-118, wherein the biological sample is systemic (whole) blood, plasma, serum, lung lavage, a cell lysate, menstrual blood, urine, a tissue sample, amniotic fluid, cerebrospinal fluid, tears, a liquid biopsy, saliva, or semen, preferably a plasma sample.

Embodiment 121: The method of any one of embodiments 113-120, wherein the biological sample is run through a depletion column or a spin column with resin.

Embodiment 122: The method of any one of embodiments 113-121, wherein
at least a 0.1-fold, 0.5-fold, 1-fold, or more increase in the number of biomarkers or patterns of biomarkers is observed compared to a biological sample without the one or more protein-binding agent and the means for binding one or more high-abundance protein and/or their proteoforms; and/or
at least a 0.1-fold, 0.5-fold, 1-fold, or more increase in the number of biomarkers or patterns of biomarkers compared to a biological sample comprising the one or more protein-binding agent without the addition of the means for binding one or more high-abundance protein and/or their proteoforms.

Embodiment 123: The method of any one of embodiments 113-122, further comprising preparing the complex for detecting the one or more biomarker or a pattern of one or more biomarker associated with the disease in the protein corona.

Embodiment 124: The method of embodiment 123 comprising:
separating the complex from the remainder of the biological sample;
washing the complex;
resuspending the complex;
reducing the proteins from the complex;
alkylating the proteins from the complex; and
digesting the proteins from the complex.

Embodiment 125: The method of any one of embodiments 113-124, wherein the disease is a neoplastic disease, cardiovascular disease, metabolic disease, infectious disease, inflammatory disease, congenital and hereditary diseases, degenerative disease, a neurological disease, or a combination thereof.

Embodiment 126: The method of embodiment 125, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.

Embodiment 127: The method of embodiment 125, wherein the disease is a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.

Embodiment 128: The method of any one of embodiments 113-127, wherein about 1 pg/ml to about 1 g/ml of protein-binding agents are added to the biological sample.

1. A method for detecting proteins and/or their proteoforms in a biological sample, the method comprising:
adding one or more small molecule to the biological sample;
adding one or more protein-binding agent having a surface capable of binding proteins to the biological sample and allowing a protein corona to form on the surface of the one or more protein-binding agent to produce a complex comprising the protein corona and the one more protein-binding agent; and
detecting the number of proteins and/or their proteoforms in the protein corona by an antibody-based technique or a proteomics technique.

2. The method of claim 1, wherein detecting proteins and/or their proteoforms in the protein corona is by a proteomics technique comprising top-down, middle-down or bottom-up LC-MS/MS.

3. The method of claim 1, wherein the one or more protein-binding agent comprises an inorganic agent, metal-based agent, metal oxide-based agent, polymer-based agent, lipid-based agent, carbon-based agent, core-shell agent, composite agent, mesoporous agent, or a combination thereof.

4. The method of claim 1, wherein the one or more protein-binding agent comprises one or more nanoscale or microscale material, comprising a nanoparticle, nanorod, nanosphere, nanodisk, nanocluster, nanofiber, nanotube, microparticle, microrod, microsphere, microbead, or a combination thereof.

5. The method of claim 1, wherein the one or more protein-binding agent has a polydispersity index (PDI) of about 0.01 to about 0.7.

6. The method of claim 1, wherein the one or more protein-binding agent is of the same type.

7. The method of claim 1, wherein the biological sample is blood, plasma, serum, lung lavage, a cell lysate, menstrual blood, urine, a tissue sample, amniotic fluid, cerebrospinal fluid, tears, a liquid biopsy, saliva, or semen.

8. The method of claim 1, wherein the one or more small molecule comprises a biomolecule.

9. The method of claim 1, wherein the one or more small molecule comprises a metabolite, a lipid, a nutrient, a plant-derived molecule, or a combination thereof.

10. The method of claim 9, wherein the one or more small molecule comprises:
a triacylglycerol and/or a derivative thereof;
a diacylglycerol, such as 1,2 and 1,3 diacylglycerol, and/or a derivative thereof;
a glycerophospholipid and/or a derivative thereof;
a combination of a triacylglycerol and/or a derivative thereof, a diacylglycerol and/or a derivative thereof, and a glycerophospholipid and/or a derivative thereof;
glucose and/or a derivative thereof;
inosine 5′-monophosphate and/or a derivative thereof;
vitamin B complex and/or a derivative thereof;
a combination of glucose and/or a derivative thereof, inosine 5′-monophosphate and/or a derivative thereof, and vitamin B complex and/or a derivative thereof;
a combination of a triacylglycerol and/or a derivative thereof, a diacylglycerol and/or a derivative thereof; a glycerophospholipid and/or derivative thereof, glucose and/or a derivative thereof, inosine 5′-monophosphate and/or a derivative thereof, and vitamin B complex and/or a derivative thereof;
phosphatidylcholine and/or a derivative thereof;
phosphatidylethanolamine and/or a derivative thereof;
phosphatidylserine and/or a derivative thereof;
phosphatidic acid and/or a derivative thereof;
phosphatidylinositol and/or a derivative thereof;
phosphatidylglycerol and/or a derivative thereof;
cardiolipin and/or a derivative thereof;
L-α-phosphatidylinositol and/or a derivative thereof;
a combination of glucose and/or a derivative thereof, a triacylglycerol and/or a derivative thereof, a diacylglycerol and/or a derivative thereof; and phosphatidylcholine and/or a derivative thereof;
a combination of phosphatidylethanolamine and/or a derivative thereof, L-α-phosphatidylinositol and/or a derivative thereof, inosine 5′-monophosphate and/or a derivative thereof, and vitamin B complex and/or a derivative thereof; or
a combination thereof.

11. The method of claim 1, wherein the one or more small molecule is phosphatidylcholine.

12. The method of claim 11, wherein about 1 pg/ml to about 1 g/ml phosphatidylcholine is added to the biological sample.

13. The method of any claim 1, wherein the biological sample is run through a depletion column or a spin column with resin.

14. The method of claim 1, wherein
at least a 0.1-fold, 0.5-fold, 1-fold, or more increase in the number of proteins and/or their proteoforms is observed compared to a biological sample without the one or more protein-binding agent and the one or more small molecule; and/or
at least a 0.1-fold, 0.5-fold, 1-fold, or more increase in the number of proteins and/or their proteoforms compared to a biological sample comprising the one or more protein-binding agent without the addition of the one or more small molecule.

15. The method of claim 1, wherein the one or more small molecule is added to the biological sample at diverse concentrations.

16. The method of claim 15, wherein the diverse concentrations are from about 1 pg/ml to about 1 g/ml.

17. The method of claim 1, further comprising preparing the complex for detecting the proteins and/or their proteoforms in the protein corona.

18. The method of claim 17 comprising:
separating the complex from the remainder of the biological sample;
washing the complex;
resuspending the complex;
reducing the proteins from the complex;
alkylating the proteins from the complex; and
digesting the proteins from the complex.

19. A method for detecting one or more biomarker or a pattern of one or more biomarker associated with a disease, the method comprising:
adding one or more small molecule to at least two biological samples each biological sample from different subjects diagnosed with the disease;
adding one or more protein-binding agent having a surface capable of binding proteins to the biological samples and allowing a protein corona to form on the surface of the one or more protein-binding agent to produce a complex comprising the protein corona and the one or more protein-binding agent; and
detecting the one or more biomarker or the pattern of one or more biomarker in the protein corona by an antibody-based technique or a proteomics technique.

20. The method of claim 19, wherein detecting the one or more biomarker or the pattern of one or more biomarker in the protein corona is by a proteomics technique comprising top-down, middle-down or bottom-up LC-MS/MS.

21. The method of claim 19, wherein the disease is a neoplastic disease, cardiovascular disease, metabolic disease, infectious disease, inflammatory disease, congenital and hereditary diseases, degenerative disease, a neurological disease, or a combination thereof.

22. The method of claim 21, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.

23. A method of diagnosing a disease in a subject, the method comprising:
adding one or more small molecule to a biological sample from the subject;
adding one or more protein-binding agent having a surface capable of binding proteins to the biological sample and allowing a protein corona to form on the surface of the one or more protein-binding agent to produce a complex comprising the protein corona and the one or more protein-binding agent; and
detecting one or more biomarker or a pattern of one or more biomarker associated with the disease in the protein corona by an antibody-based technique or a proteomics technique.

24. The method of claim 23, wherein detecting the one or more biomarker or the pattern of one or more biomarker in the protein corona is by a proteomics technique comprising top-down, middle-down or bottom-up LC-MS/MS.

25. The method of claim 23, wherein the disease is a neoplastic disease, cardiovascular disease, metabolic disease, infectious disease, inflammatory disease, congenital and hereditary diseases, degenerative disease, a neurological disease, or a combination thereof.

26. The method of claim 25, wherein the disease is a neoplastic disease selected from lung cancer, pancreas cancer, myeloma, myeloid leukemia, meningioma, glioblastoma, breast cancer, esophageal squamous cell carcinoma, gastrointestinal cancer, liver cancer, prostate cancer, bladder cancer, cervical cancer, ovarian cancer, thyroid cancer, neuroendocrine cancer, and a combination thereof.

27. The method of claim 25, wherein the disease is a neurological disease selected from Alzheimer's disease, brain tumors, epilepsy, Parkinson's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease, multiple sclerosis, Devic's disease, central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, anti-MAG peripheral neuropathy, and a combination thereof.
